(19) United States
(12) Patent Application Publication
(10) Pub. No.: US 2011/0256061 A1
(43) Pub. Date: Oct. 20, 2011

(54) Binding agents to the Integrin Alpha-11 subunit, and uses thereof

(76) Inventor: Evy Lundgren-Akerlund, Bjarred (SE)

(21) Appl. No.: 12/448,342

(22) PCT Filed: Dec. 18, 2007
(86) PCT No.: PCT/GB2007/004886

§ 371 Date: Jun. 18, 2009

Related U.S. Application Data
(60) Provisional application No. 60/875,388, filed on Dec. 18, 2006.
(60) Provisional application No. 60/875,389, filed on Dec. 18, 2006.

(30) Foreign Application Priority Data
Dec. 18, 2006 (GB) 0625144.1
Dec. 18, 2006 (GB) 0625165.6

(51) Int. Cl. A61K 49/00; C12N 15/63; C12N 1/00; C12N 5/10; C07K 16/18; C12P 21/00; A61K 39/395; C07H 21/00; C07K 14/00; A61P 29/00; A61P 19/02; A61P 1/00; A61P 17/06; A61P 11/06; A61P 37/06; A61P 25/28; G01N 33/566

(52) U.S. Cl. 424/9.2; 435/7.21; 436/501; 435/320.1; 435/243; 435/332; 530/389.1; 435/69.6; 424/172.1; 424/9.1; 536/23.53; 530/387.3; 530/388.2; 530/350

(57) ABSTRACT

The present invention relates to an antibody or antigen-binding fragment, or a variant, fusion or derivative thereof, for use in medicine, wherein the antibody or antigen-binding fragment, variant, fusion or derivative thereof has binding specificity for an integrin α11 subunit, or a heterodimer thereof. In particular, the invention relates to the use of such antibodies or antigen-binding fragments, variants, fusions or derivatives thereof, in the treatment of inflammatory disorders such as arthritic diseases (e.g. rheumatoid arthritis and osteoarthritis).

### FIELD OF THE INVENTION

The present invention relates to binding agents with binding specificity for an integrin α11 subunit, or heterodimer comprising the same (for example, an α11β1 heterodimer), and the use thereof in the treatment of an inflammatory condition. Specifically, the invention provides antibodies and antigen-binding fragments, variants, fusions and derivatives thereof, for use in the treatment of inflammatory conditions, such as arthritic diseases, and methods of using the same.

### BACKGROUND

### Integrins Regulate Diverse Events by Cell-Cell and Cell-Matrix Interactions

Integrins were originally identified as intermediary cell surface structures that linked the internal cytoskeleton with the immediate environment or extracellular cell matrix, and were considered functionally “dead” molecules. This reasoning was partially based on the observation that most integrins contain only a very small cytoplasmic tail lacking any signalling motifs. Today, integrins are known as highly complex structures that via interaction with other cell surface receptors and recruitment of intracellular adapter proteins participate in cell signalling from the inside and out (Phillips et al., 1988, Blood 71:831-43.), from outside and in (Law et al., 1999, Nature 401:808-811.), and have been shown to transduce signals laterally across the cell membrane (Hynes, 2002, Cell 110:673-87; for review see Miranti & Brugge, 2002, Nat Cell Biol 4:E83-90.).

Blocking of some of the best studied integrins using monoclonal antibodies or small molecule inhibitors has been shown to abrogate cell-cell and cell-matrix contacts resulting in intervention of diverse biological processes including development, tissue repair, angiogenesis, inflammation and haemostasis. Some of these antibodies are the subject of clinical phase trials (Shimaoka & Springer, 2003, Nat Rev Drug Discov 2:703-16).

Integrin-mediated immunomodulatory and extracellular matrix modulatory effects have thus far largely been restricted to stimulation of integrins via their native ECM ligands or anti-β1 integrin antibodies (Loeser, 2002, Biorheology 39:119-24). Such studies have, in analogy with other integrin receptors, demonstrated inside-out, outside-in, and lateral signalling participation of chondrocyte integrins.

### Rheumatoid Arthritis

Rheumatoid arthritis (RA) is an inflammatory, autoimmune disease that affects synovial joints, causing pain, swelling, and reduced mobility for the patient. All peripheral joints can be affected in RA, but the most commonly affected are those of the hands, feet and knees. In RA, the immune system attacks the synovium (tissue lining the joint capsule) for unknown reasons, causing local inflammation. The inflammation ultimately results in destruction of cartilage and bone within the joint, as well as the destruction of ligaments, tendons, and muscles that support the joint. In the rheumatoid synovium, activated T cells, B cells, macrophages, fibroblasts, endothelial cells and plasma cells can be identified. Many of the treatments in the pipeline focus on the more targeted inhibition of these specific cells. Although the actual cause of RA is unknown, there is a strong inheritance component.

RA occurs in 0.5-2.0% of the adult population worldwide with one in three patients becoming severely disabled within 20 years. The prevalence of RA is three times greater in women than in men, with a peak age of onset usually between 20 to 45 years. Mortality rates are 27% higher in RA sufferers than in age- and gender-matched controls and even higher in the subset of women. This translates to a reduced life expectancy of somewhere between 5 to 18 years depending on the study. Radiographically evident joint disease is seen in >67% of patients with the first 2 years and >72% of patients within the first 5 years.

RA is a chronic, systemic inflammatory disease that targets synovial joints and is often accompanied by an array of extra-articular manifestations. The arthritis is characterized by a multicellular inflammatory process of infiltration of lymphocytes and granulocytes into the articular cartilage, proliferation of synovial fibroblast and macrophages and neovascularization of the synovial lining surrounding the joint. This proliferative process induces swelling, erythema and pain of the joints, but also progress into destruction of the joint and loss of bone density and architecture.

The pathological mechanisms of RA are not fully understood. It is believed, however, that many cell types, such as macrophages, dendritic cells, fibroblast like synoviocytes, mast cells, T cells and B cells are involved. The release of cytokines such as TNF-α, IL-1, IL-6, IL-8, IL-15 and various chemokines is substantial in the RA synovium. The levels of TNF-α and IL-1 strongly correlate with disease symptoms and joint damage. Levels of auto-antibodies, such as rheumatoid factor (RF) and anti-citrulline antibodies in blood is characteristic for RA and provide diagnostic and prognostic markers for the disease.

Rheumatoid arthritis is initially characterised by an inflammatory response of the synovial membrane conveyed by an influx of a number of different cell types. The lining becomes hyperplastic and expands. In addition, bone destruction is seen.

Disease-modifying drugs are available in RA, but they are of limited use due to side-effects, and many patients do not respond to existing therapy. During the past 20 years a more aggressive approach to the treatment of RA has led to the development of disease-modifying antirheumatic drugs (DMARDS) that attempt to halt disease progression. The most prescribed DMARD is methotrexate, originally used in the treatment of cancer. Others include gold salts, antimalarials, sulphasalazine, tetracyclines and cyclosporine. Physicians usually prescribe DMARDs at disease onset while moderate-to-severe RA suffers are given NSAIDs, corticosteroids and DMARDs concurrently.

Although methotrexate is effective in the majority of RA patients, many cannot tolerate the side effects of the drug, and roughly 50% of patients eventually fail treatment, subsequently becoming eligible for more recent treatment innovations, such as biologic response modifiers. Leflunomide (Arava; Aventis) which was launched in 1998, is similar in mechanism and side effects to methotrexate and is used as a second-line agent in patients that have failed methotrexate therapy. The most successful of the novel RA therapies are the tumour-necrosis factor-α (TNFα) inhibitors, such as etanercept (Enbrel; Amgen), infliximab (Remicade; J&J/Centocor) and adalimumab (Humira; Abbott). The TNFα-inhibitors, however, also have potential side effects such as increased infections and cancer in long term use.

### Osteoarthritis

Osteoarthritis (OA) is a progressive, degenerative joint disease and is the most common form of arthritis. It strongly associates with aging and is a major cause of pain and disability in the elderly. A variety of mechanical, metabolic or constitutional insults may trigger OA. Often the insults remain unclear (‘primary’ OA) but sometimes a clear cause such as trauma may be apparent (‘secondary’ OA). All the joint tissues (cartilage, bone, synovium, capsule, ligament, muscle) depend on each other for health and function. Insult to one impacts on the others resulting in a common OA phenotype affecting the whole joint. The OA process involves new tissue production, most notably bone (‘osteophyte’), and remodelling of joint shape. Often OA compensates for the insults, resulting in an anatomically altered but pain-free functioning joint (‘compensated’ OA). Sometimes, however, it fails, resulting in progressive damage, associated symptoms and presentation as an OA patient with ‘joint failure’. Such a perspective explains the clinical heterogeneity of OA and the variable clinical outcomes.

Osteoarthritis is the leading cause of physical disability over the age of 65 years affecting an estimated 10% of the population. The prevalence of OA-related physical disability is greater in women than men and rises steadily to 25% in women over the age of 85 years. Predictions suggest that there will be a 66% increase in the number of people with OA-related disability by the year 2020. A similar increase in the number of people with severe symptomatic OA of the hip and knee requiring joint replacement surgery in the next 30 years is predicted if disease-modifying strategies for the medical treatment and prevention of OA cannot be found.

Osteoarthritis (OA) is not a single disease or process but rather the clinical and pathological outcome of a range of processes and disorders that lead to structural, and eventually symptomatic, failure of one or more synovial joints. OA involves the entire joint including the subchondral bone, ligaments, capsule, synovial membrane and periarticular muscles as well as the articular cartilage. Ultimately the articular cartilage degenerates with fibrillation, fissures, ulceration and full thickness loss of the joint surface.

Treatment of osteoarthritis includes a wide spectrum of approaches where most treatments are palliative with the exception of surgery. This means that most treatments relieve pain and thereby increase joint function of the patient, but the treatments do not change the course of the disease. Surgical interventions include joint replacement and osteotomy, which may reverse the progress of osteoarthritis and provide long-term improved function and pain relief for a specific joint.

There is thus also an urgent need to improve the options to treat OA. Existing drug therapies for OA reduce the symptoms (mainly pain), and are only moderately effective and often leave patients with a substantial pain burden. Thus, to date, no drugs are available with proven disease-modifying efficacy in OA.

Accordingly, the present invention seeks to provide novel treatments for inflammatory conditions such as RA and OA, and novel binding agents for use in the same.

### SUMMARY OF THE INVENTION

A first aspect of the present invention provides the use of an antibody or an antigen-binding fragment thereof with binding specificity for an integrin α11 subunit, or a heterodimer thereof, or of a variant, fusion or derivative of said antibody or antigen binding fragment, or a fusion of a said variant or derivative thereof, which retains the binding specificity of said antibody or an antigen-binding fragment thereof for an integrin α11 subunit, or a heterodimer thereof, in the preparation of a medicament for treating an inflammatory condition, wherein the antibody or fragment comprises the following amino acid sequences:

| | |
|---|---|
| FSRYYMHWVRQVPG; | [SEQ ID NO: 1] |
| SGVSWNGSRTHYADSVKGR; | [SEQ ID NO: 2] |
| ARVSGDGYNFGA; | [SEQ ID NO: 3] |
| CTGSSSNIGAGYDVH; | [SEQ ID NO: 4] |
| GYNERPS; | [SEQ ID NO: 5] |
| and | |
| CAAWDDSLSGHVV. | [SEQ ID NO: 6] |

It will be appreciated by persons skilled in the art that the above amino acid sequences of SEQ ID NOS:1 to 6 represent complementarity determining regions (CDRs).

A second aspect of the invention provides the use of an antibody or antigen-binding fragment, or variant, fusion or derivative thereof, as described above in the preparation of a diagnostic or prognostic agent for an inflammatory condition.

A third aspect of the invention provides the use of an antibody or antigen-binding fragment, or variant, fusion or derivative thereof, as described above in the preparation of an agent for detecting and/or imaging cells expressing an integrin α11 subunit, or heterodimer thereof.

Related aspects of the invention provide:
(a) the use of an antibody or antigen-binding fragment, or variant, fusion or derivative thereof, as described above for treating an inflammatory condition;
(b) the use of an antibody or antigen-binding fragment, or variant, fusion or derivative thereof, for diagnosing or prognosing an inflammatory condition; and
(c) the use of an antibody or antigen-binding fragment, or variant, fusion or derivative thereof, for detecting and/or imaging cells expressing an integrin α11 subunit, or heterodimer thereof.

By “binding specificity” for an integrin α11 subunit, or a heterodimer thereof, we mean an antibody or antigen-binding fragment, or variant, fusion or derivative thereof, which is capable of binding to an integrin α11 subunit, or a heterodimer thereof such as an α11β1 heterodimer.

In one embodiment, the integrin α11 subunit, or the heterodimer thereof, is localised on the surface of a cell, such as a chondrocyte, a fibroblast, a mesenchymal cell (e.g. a mesenchymal stem cell) or a dendritic cell.

In a further embodiment, the antibody or antigen-binding fragment, or variant, fusion or derivative thereof, is capable of binding to an integrin α11 subunit, or a heterodimer thereof in vivo, i.e. under the physiological conditions in which an integrin α11 subunit exists inside the body. Such binding specificity may be determined by methods well known in the art, such as e.g. ELISA, immunohistochemistry, immunoprecipitation, Western blots, chromatography and flow cytometry using transfected cells expressing the α11 subunit or a heterodimer thereof (see Examples). Examples of how to measure specificity of an antibody is given in e.g. Harlow & Lane, “Antibodies: A Laboratory”, Cold Spring Habor Laboratory Press, New York, which is incorporated herein by reference.

It will be appreciated by persons skilled in the art that the integrin α11 subunit may be a human or animal integrin α11 subunit. In one embodiment, the integrin α11 subunit is human. Examples of known integrin α11 subunits are disclosed in International Patent Application (Publication) No. WO 00/75187 (to Cartela AB), which is incorporated herein by reference.

In another embodiment, the antibody or antigen-binding fragment, or variant, fusion or derivative thereof, is capable of binding to an integrin α11 subunit, or a heterodimer thereof, selectively. By “capable of binding selectively” we include such antibody-derived binding moieties which bind at least 10-fold more strongly to integrin α11 subunit or a heterodimer thereof than to another proteins (in particular other integrins, such as α10, α1 and α2 having most identity with α11); for example at least 50-fold more strongly or at least 100-fold more strongly. The binding moiety may be capable of binding selectively to integrin α11 subunit or a heterodimer thereof under physiological conditions, e.g. in vivo. Suitable methods for measuring relative binding strengths include immunoassays, for example where the binding moiety is an antibody (see Harlow & Lane, “Antibodies: A Laboratory”, Cold Spring Habor Laboratory Press, New York, which is incorporated herein by reference). Alternatively, binding may be assessed using competitive assays or using Biacore® analysis (Biacore International AB, Sweden).

In a further embodiment, the antibody or antigen-binding fragment, or variant, fusion or derivative thereof, binds exclusively to an integrin α11 subunit or a heterodimer thereof.

It will be appreciated by persons skilled in the art that the binding specificity of an antibody or antigen binding fragment thereof is conferred by the presence of complementarity determining regions (CDRs) within the variable regions of the constituent heavy and light chains. In the uses of the present invention, binding specificity for an integrin α11 subunit, or a heterodimer thereof, is conferred by the presence of the CDRs identified as SEQ ID NOS: 1 to 6 above, or variant sequences thereof which retain the same binding specificity.

By “retain the binding specificity” we mean that the antibody or antigen-binding fragment, or variant, fusion or derivative thereof, is capable of competing for binding to an integrin α11 subunit with antibody ‘A03’ (see SEQ ID NO: 11 and Examples below).

For example, the antibody or antigen-binding fragment, or variant, fusion or derivative thereof, may bind to the same epitope on the integrin α11 subunit as an antibody comprising the CDRs identified as SEQ ID NOS: 1 to 6.

By “epitope” it is herein intended to mean a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. way which helices or sheets are folded or arranged to give a three-dimensional structure, of an antigen.

An example of a suitable epitope (or part thereof) of the integrin alpha11 subunit is as follows:

[SEQ ID NO: 7]
WNGCNEDEHCVPDLVLDARSDLPTAMEYCQRVLRKPAQDCSAYTLSF
DTTVFTIESTRQRVAVEATLENRGENAYSTVLNISQSANLQFASLIQKED
SDGSIECVNEERRLQKQVCNVSYPFFRAKAKVAFRLDFEFSKSIFLHHLE
IELAAGSDSNERDSTKEDNVAPLRFHLKYEADVLFTRSSSLSHYEVKLN
SSLERYDGIGPPFSCIFRIQNLGLFPIHGMMMKITIPIATRSGNRLLKLR
DFLTDEANTSCNIWGNSTEYRPTPVEEDLRRAPQLNHSNSDVVSINCNIR
LVPNQEINFHLLGNLWLRSLKALKYKSMKIMVNAALQRQFHSPFIFREED
PSRQIVFEISKQEDWQVP

Further suitable epitopes of integrin alpha11 may include fragments, fusions or natural variants of SEQ ID NO: 7.

For example, suitable fragments comprise or consist of from about amino acid number 778 to amino acid number 1142 of the human alpha 11 integrin sequence, as shown in SEQ ID NO: 7, or the extracellular extension region, such as a peptide comprising essentially the amino acid sequence from about amino acid number 804 to about amino acid number 826 of the human alpha 11 integrin sequence, especially a peptide comprising essentially the amino acid sequence EYCQRVLRKPAQDCSAYTLSFDT [SEQ ID NO: 8], or any poly peptide fragment that comprises, or consists of, about amino acid amino acid (aa) numbers: aa778- to aa800, about aa no 800-820, about aa no 820-840, about aa no 840-860, about aa no 860-880, about aa no 880-900, about aa no 900-920, about aa no 920-940, about aa no 940-960, about aa no 960-980, about aa no 980-1000, about aa no 1020-1040, about aa no 1040-1060, about aa no 1080-1100, about aa no 1100-1120, about aa no 1120-1142 of the human alpha11 sequence.

All amino acid numbers above are calculated from the human alpha11 sequence amino acid numbers where amino acid no 1 is M, aa no 2 is D, aa no 3 is L, aa no 4 is P, aa no 5 is R, aa no 6 is G and aa no 7 is L, etc. of the human integrin alpha11 as disclosed in WO 00/75187 (which is incorporated herein by reference).

Suitable epitope fusions include a fusion of the above peptide to a StrepTag tail.

Epitope variants, such as natural variants, of the above epitope polypeptide sequence include polypeptides comprising a sequence with at least 60% identity to the amino acid sequence of SEQ ID NO: 7, preferably at least 70% or 80% or 85% or 90% identity to said sequences, and more preferably at least 95%, 96%, 97%, 98% or 99% identity to said amino acid sequences.

Percent identity can be determined by methods well known in the art, for example using the LALIGN program (Huang and Miller, Adv. Appl. Math. (1991) 12:337-357) at the Expasy facility site:

(http://www.ch.embnet.org/software/LALIGN_form.html)
using as parameters the global alignment option, scoring matrix BLOSUM62, opening gap penalty—14; extending gap penalty—4.

Alternatively, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The antibody or antigen-binding fragment, or variant, fusion or derivative thereof, which retains the binding specificity of antibody ‘A03’ may also retain one or more of the same biological properties as antibody ‘A03’ (see below).

Methods for determining whether a test antibody is capable of competing for binding with second antibody are well known in the art.

One suitable method is a sandwich ELISA for evaluation of the α11-binding antibody, A03. A suitable amount of, e.g. a polyclonal antibody, such as 10 μg/ml of a polyclonal rabbit antibody, directed against the cytoplasmatic tail of human alpha11. The antibody is then used as a capture antibody when coated in a 96-well plate, such as e.g. Maxisorp Nunc™. Coating is done according to standard procedure known in the art, e.g. by incubation the plate at 4° C. over night, followed by three times washing with e.g. TBS-T (20 mM Tris-HCl pH 7.5, 150 mM NaCl and 0.05% Tween-20). Wells may be blocked for 1 h with 3% BSA in TBS-T at e.g. room temperature. Cell extract from HEK-cells stably transfected with human alpha11 expression vector, is then diluted in assay buffer (e.g. TBS-T supplemented with 0.1% BSA, 1 mM MgCl2 and 10 μM CaCl₂). Suitable amount of diluted cell extract, such as e.g. 50 μl is then added per well and incubated to allow binding to the coated antibody, e.g. for 1 h at room temperature. Plates are then washed three times with TBS-T. Primary antibody (A03 or control antibody) conjugated with biotin is then added in a suitable amount, such as e.g. at 2 μg/ml in assay buffer. Plates are then incubated for a sufficient time to allow binding of A03 and control antibody where possible, e.g. for 1 h at room temperature followed by three washes in TBS-T. In case where biotinylated primary antibodies are used, Streptavidin-HRP antibody (DAKO) may be used and diluted accordingly in assay buffer (1:5000), added to the wells. Plates are then incubated enough for the streptavidin-biotin complex to form, e.g. for 1 h at room temperature. Following washing, e.g. three times with TBS-T and the plates are developed with peroxidase substrate (e.g. OPD SigmaFast, Sigma). The absorbance of the colorimetric change is determined at suitable wavelength, in this case 490 nm.

If primary antibodies are not conjugated, the same ELISA can instead be incubated with a secondary antibody against the human IgG4 directly conjugated with HRP, e.g. mouse anti-human IgG4-HRP, from e.g. Serotec, or, if not conjugated, followed by a HRP-conjugated anti-mouse antibody from e.g. DAKO. Plates are then washed and developed as outlined above.

Further methods include reversing the sandwich ELISA outlined above, and instead use the α11 antibody A03 as a capture antibody. In this case, cell extracts containing α11 is added and the ELISA is preceded as outlined above with the following modifications:

As primary antibody the polyclonal rabbit antibody directed against the cytoplasmic tail of human alpha11 is diluted in assay buffer (1 μg/ml). As a secondary antibody a HRP-conjugated goat anti-rabbit antibody may be used (e.g. DAKO, diluted 1:5000). Plates are then washed and developed as outlined above.

In a further ELISA assay, it is possible to evaluate epitope-modifying or blocking antibodies. This can be done in several ways, for instance using the sandwich ELISA detailed above with the following modifications:

Capture antibody as well as alpha11 is added to the 96-well plate as detailed above. Before applying primary antibody, plates are incubated with a test antibody, e.g. epitope-modifying or blocking antibodies at 10 μg/ml in assay buffer. Plates are incubated for 15 min at room temperature after which primary antibody (A03 or control antibody) conjugated with biotin is added and plates are incubated for 1 h at room temperature. Plates are washed and developed as outlined above.

The reverse sandwich ELISA can also be used to evaluate a test antibody, e.g. an epitope-modifying or blocking antibody. In this ELISA, the alpha11 antibody A03 can be used as a capture antibody. Cell extract containing alpha11 is then added as outlined above and different conjugated, potentially modifying antibodies, can be used as primary antibodies.

A further method to evaluate if a test antibody is capable of competing for binding with second antibody is by flow cytometry, such as Fluorescence Activated Cell Sorting (FACS®) as described in Example IV below.

Additional methods suitable for identifying competing antibodies are disclosed in Antibodies: A Laboratory Manual, Harlow & Lane, Cold Spring Habor Laboratory Press, New York, ISBN 0-87969-314-2 which is incorporated herein by reference (for example, see pages 567 to 569, 574 to 576, 583 and 590 to 612).

By “antibody” we include substantially intact antibody molecules, as well as chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same. For example, the antibody may be a monoclonal antibody.

Thus, in one embodiment the antibody or antigen-binding fragment, or variant, fusion or derivative thereof, comprises or consists of an intact antibody.

For example, the antibody or antigen-binding fragment, or a variant, fusion or derivative thereof, may consist essentially of an intact antibody. By “consist essentially of” we mean that the antibody or antigen-binding fragment, variant, fusion or derivative thereof consists of a portion of an intact antibody sufficient to retain binding specificity for an integrin α11 subunit.

The term ‘antibody’ also includes all classes of antibodies, including IgG, IgA, IgM, IgD and IgE. In one embodiment, however, the antibody is an IgG molecule, such as an IgG1, IgG2, IgG3, or IgG4 molecule.

In one embodiment, the antibody is an IgG4 molecule.

In a further embodiment, the antibody is a non-naturally occurring antibody. Of course, where the antibody is a naturally occurring antibody, it is provided in an isolated form (i.e. distinct from that in which it is found in nature).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison et al (1984) Proc. Natl. Acad. Sci. USA 81, 6851-6855).

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature. 341, 544). A general review of the techniques involved in the synthesis of antibody fragments which retain their specific binding sites is to be found in Winter & Milstein (1991) Nature 349, 293-299.

Thus, by “antigen-binding fragment” we mean a functional fragment of an antibody that is capable of binding to an integrin α11 subunit, or a heterodimer thereof.

Exemplary antigen-binding fragments may be selected from the group consisting of Fv fragments (e.g. single chain Fv and disulphide-bonded Fv), Fab-like fragments (e.g. Fab fragments, Fab′ fragments and F(ab)₂ fragments), single antibody chains (e.g. heavy or light chains), single variable domains (e.g. V_{H} and V_{L} domains) and domain antibodies (dAbs, including single and dual formats [i.e. dAb-linker-dAb]).

In one embodiment, the antigen-binding fragment is an scFv.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

Also included within the scope of the invention are modified versions of antibodies and an antigen-binding fragments thereof; e.g. modified by the covalent attachment of polyethylene glycol or other suitable polymer, and uses of the same.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299, which are incorporated herein by reference) or generation of monoclonal antibody molecules by cell lines in culture. These include, but are not limited to, the hybridoma technique, the human B-cell hybridoma technique, and the Epstein-Barr virus (EBV)-hybridoma technique (see Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120, which are incorporated herein by reference).

The antibody or antigen-binding fragment or derivative thereof may be produced by recombinant means.

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, humanised antibodies may be used. Humanised forms of non-human (e.g. murine) antibodies are genetically engineered chimaeric antibodies or antibody fragments having preferably minimal-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567, which are incorporated herein by reference) by substituting human complementarity determining regions with corresponding rodent complementarity determining regions. Accordingly, such humanised antibodies are chimaeric antibodies, wherein substantially less than an intact human variable, domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95, Soderlind et al., 2000, Nat Biotechnol 18:852-6 and WO 98/32845 which are incorporated herein by reference).

Once suitable antibodies are obtained, they may be tested for activity, such as binding specificity or a biological activity of the antibody, for example by ELISA, immunohistochemistry, flow cytometry, immunoprecipitation, western blots, etc. The biological activity may be tested in different assays with readouts for that particular feature. Examples of one or more biological activity of the antibodies according to the invention are:
a) protection of cartilage from degradation (e.g. reduction in proteoglycan content);
b) reduction in inflammation of the joints (e.g. swelling); and/or
c) reduction in infiltration of inflammatory cells.

Examples of suitable assays for testing said biological activity is known in the art and also given in the examples herein.

In one embodiment of the first aspect of the invention, the antibody or antigen-binding fragment, or variant, fusion or derivative thereof, comprises a heavy chain variable region comprising the CDRs identified by SEQ ID NOS: 1 to 3. For example, the heavy chain variable region may comprise, consist essentially of or consist of the amino acid sequence of SEQ ID NO: 9:

[SEQ ID NO: 9]
EVQLLESGGGLVQPGGSLRLSCAASGFTFSRYYMHWVRQVPGKGLEW
VSGVSWNGSRTHYADSVKGRFTISRDNSKNTLYLQMNSLRAEDTAVY
YCARVSGDGYNFGAWGQGTLVTVSS

In a further embodiment of the first aspect of the invention, the antibody or antigen-binding fragment, or variant, fusion or derivative thereof, comprises a light chain variable region comprising the CDRs identified by SEQ ID NOS: 4 to 6. For example, the light chain variable region may comprise, consist essentially of or consist of the amino acid sequence of SEQ ID NO: 10:

[SEQ ID NO: 10]
QSVLTQPPSASGTPGQRVTISCTGSSSNIGAGYDVHWYQQLPGTAPKLLI
YGYNERPSGVFDRFSGSKSGTSASLAISGLRSEDEADYYCAAWDDSLSG
HVVFGGGTKLTVLG

Thus, in one embodiment, the antibody or antigen-binding fragment, or variant, fusion or derivative thereof, comprises a heavy chain variable region comprising, consisting essentially of or consisting of the amino acid sequence of SEQ ID NO: 9 and a light chain variable region comprising, consisting essentially of or consisting of the amino acid sequence of SEQ ID NO: 8.

For example, the antibody or antigen-binding fragment, or variant, fusion or derivative thereof, may comprise a heavy chain comprising, consisting essentially of or consisting of the amino acid sequence of SEQ ID NO: 11 and/or a light chain comprising, consisting essentially of or consisting of the amino acid sequence of SEQ ID NO: 12:

[SEQ ID NO: 11]
EVQLLESGGGLVQPGGSLRLSCAASGFTFSRYYMHWVRQVPGKGLEW
VSGVSWNGSRTHYADSVKGRFTISRDNSKNTLYLQMNSLRAEDTAVY
YCARVSGDGYNFGAWGQGTLVTVSSASTKGPSVFPLAPCSRSTSESTAA
LGCLVKDYFPEPVTVSWNSGALTSGVHTFPAVLQSSGLYSLSSVVTVPS
SSLGTKTYTCNVDHKPSNTKVDKRVESKYGPPCPSCPAPEFLGGPSVFL
FPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHY
TQKSLSLSLGK
[SEQ ID NO: 12]
QSVLTQPPSASGTPGQRVTISCTGSSSNIGAGYDVHWYQQLPGTAPKLLI
YGYNERPSGVPDRFSGSKSGTSASLAISGLRSEDEADYYCAAWDDSLSG
HVVFGGGTKLTVLGQPKAAPSVTLFPPSSEELQANKATLVCLISDFYPG
AVTVAWKADSSPVKAGVETTTPSKQSNNKYAASSYLSLTPEQWKSHRS
YSCQVTHEGSTVEKTVAPTECS

In a further exemplary embodiment, the antibody or antigen-binding fragment, or variant, fusion or derivative thereof, comprises or consists of the amino acid sequence of SEQ ID NO:13.

[SEQ ID NO: 13]
EVQLLESGGGLVQPGGSLRLSCAASGFTFSRYYMHWVRQVPGKGLEW
VSGVSWNGSRTHYADSVKGRFTISRDNSKNTLYLQMNSLRAEDTAVY
YCARVSGDGYNFGAWGQGTLVTVSSGGGGSGGGGSGGGGSQSVLTQP
PSASGTPGQRVTISCTGSSSNIGAGYDVHWYQQLPGTAPKLLIYGYNER
PSGVPDRFSGSKSGTSASLAISGLRSEDEADYYCAAWDDSLSGHVVFGG
GTKLTVLG

This sequence corresponds to the exemplary ‘A03’ scFv molecule for use in the methods of the invention (the linker sequence is underlined). Optionally, the sequence may also comprise a c-myc and/or histidine tags at the C-terminus, for example:

| | |
|---|---|
| EQKLISEEDLSGSAAAHHHHHH | [SEQ ID NO: 14] |

The term ‘amino acid’ as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptide binding agents described herein comprise or consist of L-amino acids.

It will be appreciated by persons skilled in the art that invention encompasses variants, fusions and derivatives of the defined polypeptides, and fusions of said variants or derivatives, as well as uses thereof, provided that such variants, fusions and derivatives retain binding specificity for an integrin α11 subunit or heterodimer thereof.

Variants may be made using the methods of protein engineering and site-directed mutagenesis well known in the art using the recombinant polynucleotides (see example, see Molecular Cloning: a Laboratory Manual, 3rd edition, Sambrook & Russell, 2001, Cold Spring Harbor Laboratory Press, which is incorporated herein by reference).

By ‘fusion’ of said polypeptide we include a polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any variant or derivative of said polypeptide are also included in the scope of the invention. It will be appreciated that fusions (or variants or derivatives thereof) which retain desirable properties, such as retain binding specificity for an integrin α11 subunit or heterodimer thereof, are preferred.

The fusion may comprise a further portion which confers a desirable feature on the said polypeptide of the invention; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

By ‘variants’ of the polypeptide we include insertions, deletions and substitutions, either conservative or non-conservative. In particular we include variants of the polypeptide where such changes do not substantially alter the activity of the said polypeptide. For example, we include variants of the polypeptide where such changes do not substantially alter the binding specificity for an integrin α11 subunit or heterodimer thereof.

The polypeptide variant may have an amino acid sequence which has at least 75% identity with one or more of the amino acid sequences given above, for example at least 80%, at least 90%, at least 95%, at least 96%, at least 97%, at least 98%, at least 99%, or 100% identity with one or more of the amino acid sequences specified above.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680).

The parameters used may be as follows:
Fast pairwise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The polypeptide, variant, fusion or derivative of the invention may comprise one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-allyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which are capable of binding an integrin α11 subunit. The term ‘peptidomimetic’ refers to a compound that mimics the conformation and desirable features of a particular peptide as a therapeutic agent.

For example, the polypeptides of the invention include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, which is incorporated herein by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond.

It will be appreciated that the polypeptide may conveniently be blocked at its N- or C-terminus so as to help reduce susceptibility to exoproteolytic digestion.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary polypeptides comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. For example, the peptides of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods are discussed and disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of, the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification and is preferred because of the stable structures formed by cyclisation and in view of the biological activities observed for cyclic peptides.

Thus, in one embodiment the polypeptide binding moiety is cyclic. However, in an alternative embodiment, the polypeptide is linear.

The present invention also includes compositions comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the compounds according to the present invention.

The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The polypeptides described herein may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide loses no more than about 20%, or no more than about 25%, or no more than about 30%, or no more than about 35%, or no more than about 40%, or no more than about 45%, or no more than about 50% of its activity (prior to lyophilisation) when rehydrated.

It will be further appreciated by persons skilled in the art that the antibodies and 20, antigen-binding fragments, variants, fusions and derivatives thereof, described herein may exist in monomeric form or in the form of a homo- or hetero-multimer thereof (e.g. dimer, trimer, tetramer, pentamer, etc.).

In a further embodiment of the invention, the antibody or antigen-binding fragment, or variant, fusion or derivative thereof, comprises a therapeutic and/or detectable moiety.

By a “detectable moiety” we include the meaning that the moiety is one which, when located at the target site following administration of the compound of the invention into a patient, may be detected, typically non-invasively from outside the body and the site of the target located. The detectable moiety may be a single atom or molecule which is either directly or indirectly involved in the production of a detectable species. Thus, the binding agents of this embodiment of the invention are useful in imaging and diagnosis.

Suitable detectable moieties are well known in medicinal chemistry and the linking of these moieties to polypeptides and proteins is well known in the art. Examples of detectable moieties include, but are not limited to, the following: radioisotopes (e.g. ³H, ¹⁴C, ³⁵S, ¹²³I, ¹²⁵I, ¹³¹I, ⁹⁹Tc, ¹¹¹In, ⁹⁰Y, ¹⁸⁸Re), radionuclides (e.g. ¹¹C, ¹⁸F, ⁶⁴Cu), fluorescent labels (e.g. FITC, rhodamine, lanthanide phosphors, carbocyanine), enzymatic labels (e.g. horseradish peroxidase, β-galactosidase, luciferase, alkaline phosphatase), chemiluminescent, biotinyl groups and predetermined polypeptide epitopes recognised by a secondary reporter (e.g. leucine zipper pair sequences, binding sites for secondary antibodies, metal binding domains, epitope tags). In some embodiments, labels are attached by spacer arms of various lengths to reduce potential steric hindrance.

The radio- or other labels may be incorporated in the polypeptides of the invention in known ways. For example, if the binding moiety is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57, which is incorporated herein by reference) can be used to incorporate ¹²³I. Reference (“Monoclonal Antibodies in Immunoscintigraphy”, J-F Chatal, CRC Press, 1989, which is incorporated herein by reference) describes other methods in detail.

The antibodies and antigen-binding fragments, or variants, fusions or derivatives thereof, as described above have efficacy in the treatment of an inflammatory condition.

By ‘treatment’ we include both therapeutic and prophylactic treatment of a subject/patient. The term ‘prophylactic’ is used to encompass the use of a polypeptide or formulation described herein which either prevents or reduces the likelihood of an inflammatory condition in a patient or subject.

For example, the antibody or antigen-binding fragment, or variant, fusion or derivative thereof, may have efficacy in the treatment of an inflammatory condition selected from the group consisting of arthritic diseases (such as rheumatoid arthritis and osteoarthritis), joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and autoimmune chronic inflammatory diseases.

In one embodiment, the antibody or antigen-binding fragment, or variant, fusion or derivative thereof, has efficacy in the treatment an arthritic disease, such as rheumatoid arthritis or osteoarthritis. Such efficacy may be determined in suitable animal models, such as arthritis models in mice (see Examples).

For example, the antibody or antigen-binding fragment, or variant, fusion or derivative thereof, may be capable of modulating (e.g. inhibiting) the degradation of collagen, in vitro and/or in vivo.

By “modulate” we include increasing or decreasing the rate of degradation of collagen. It will be appreciated that inhibition of the degradation of collagen may be in whole or in part. For example, collagen degradation may be inhibited by 10% or more, e.g. 20%, 30%, 40%, 50%, 60%, 70%, 80%, 90% or 100%, compared to the degradation of collagen in the absence of the antibody or antigen-binding fragment, or variant, fusion or derivative thereof.

The antibody or antigen-binding fragment, or variant, fusion or derivative thereof, may exhibit one or more of the following medically useful properties:
a) protection of cartilage from degradation (e.g. reduction in proteoglycan content);
b) reduction in inflammation of the joints (e.g. swelling); and/or
c) reduction in infiltration of inflammatory cells.

Methods of testing for the above properties are described in the Examples below.

Hence, the antibody or antigen-binding fragment, or variant, fusion or derivative thereof, may have advantageous properties over established therapies for arthritic disease, which reduce swelling (NSAIDS) or swelling and cell infiltration (anti-TNF antibodies) in the same animal model, but are not able to affect all three parameters.

Thus, the invention provides the following methods:
a) a method for protecting cartilage from degradation comprising administering to the site of, the cartilage an antibody or antigen-binding fragment, or variant, fusion or derivative thereof, as described above;
b) a method for reducing joint inflammation comprising administering to the joint an antibody or antigen-binding fragment, or variant, fusion or derivative thereof, as described above; and
c) a method for reducing the infiltration of inflammatory cells into a joint comprising administering to the joint an antibody or antigen-binding fragment, or variant, fusion or derivative thereof, as described above.

In particular, a fourth aspect of the invention provides a method for treating an individual with an inflammatory condition, the method comprising administering to the individual an effective amount of an antibody or antigen-binding fragment, or a variant, fusion or derivative thereof, as defined above.

A fifth aspect of the invention provides a method for diagnosing or proposing an inflammatory condition in an individual, the method comprising administering to the individual an effective amount of an antibody or antigen-binding fragment, or a variant, fusion or derivative thereof, as defined above.

A sixth aspect describes a method for imaging cells expressing an integrin all subunit associated with an inflammatory condition, or heterodimer thereof, in the body of an individual, the method comprising administering to the individual an effective amount of an antibody or antigen-binding fragment, or a variant, fusion or derivative thereof, as defined above.

In one embodiment of the above aspects of the invention, the method further comprises the step of detecting the location of the compound in the'individual.

A seventh aspect of the invention provides a method for monitoring the progression of an inflammatory condition in an individual, the method comprising:
(a) providing a sample of cells collected from the individual at a first time point and measuring the amount of integrin α11 subunit protein therein using an antibody or antigen-binding fragment, or a variant, fusion or derivative thereof, as described herein;
(b) providing a sample of cells collected from the individual at a second time point and measuring the amount of integrin α11 subunit protein therein using an antibody or antigen-binding fragment, or a variant, fusion or derivative thereof, as described herein; and
(c) comparing the amount of integrin α11 subunit protein measured in steps (a) and (b)
   wherein an increased amount of integrin α11 subunit protein measured in step (b) compared to step (a) is indicative of a progression in the inflammatory condition.

An eighth aspect of the invention provides a method for identifying cells associated with an inflammatory condition, the method comprising measuring the amount of integrin α11 subunit protein in a sample of cells to be tested using an antibody or antigen-binding fragment, or a variant, fusion or derivative thereof, as described herein and comparing it to the amount of integrin α11 subunit protein to a positive and/or negative control. The positive control may comprise cells from a subject who is suffering from the inflammatory condition and the negative control may comprise cells from a healthy subject who is not suffering from the inflammatory condition.

In one embodiment of the invention, the cells are selected from the group consisting of chondrocyte cells, fibroblast cells and mesenchymal cells (e.g. mesenchymal stein cells).

The amount of integrin α11 subunit in a sample may be determined using methods well known in the art. Suitable methods for assaying integrin all protein levels in a biological sample include antibody-based techniques. For example, integrin α11 protein expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g. with urea and neutral detergent, for the liberation of integrin α11 protein for western blot or dot/slot assay (Jalkanen et al., 1985, J. Cell. Biol. 101:976-985; Jalkanen et al., 1987, J. Cell. Biol. 105:3087-3096, which are incorporated herein by reference). In this technique, which is based on the use of cationic solid phases, quantitation of integrin α11 protein can be accomplished using isolated integrin α11 protein as a standard. This technique can also be applied to body fluids.

In one embodiment, the cells to be tested are identified as cells associated with an inflammatory condition by the upregulation of integrin α11 subunit protein levels compared to corresponding normal healthy cells. By “upregulated” we mean that the integrin α11 subunit protein is increased by at least 10% compared to expression of the integrin in normal (healthy) cells. For example, the level of the integrin α11 subunit protein may be increased by at least 20%, 30%, 40%, 50%, or even 100% or more.

In a further embodiment, the above methods further comprise the step of detecting the location of the compound in the individual.

Detecting the compound or antibody can be achieved using methods well known in the art of clinical imaging and diagnostics. The specific method required will depend on the type of detectable label attached to the compound or antibody. For example, radioactive atoms may be detected using autoradiography or in some cases by magnetic resonance imaging (MRI) as described above.

In a further embodiment of the above methods of the invention, the inflammatory condition is selected from the group consisting of arthritic diseases (such as rheumatoid arthritis and osteoarthritis), joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and autoimmune chronic inflammatory diseases.

For example, the inflammatory condition may be an arthritic disease, such as rheumatoid arthritis or osteoarthritis.

A ninth aspect of the invention provides an antibody or antigen-binding fragment thereof capable of competing for binding to an integrin α11 subunit, or a heterodimer thereof, with an scFv molecule having the amino acid sequence of SEQ ID NO: 11, or a variant, fusion or derivative of said antibody or antigen-binding fragment, or a fusion of a said variant or derivative thereof, which retains the binding specificity for an integrin α11 subunit, or a heterodimer thereof, with the proviso that the antibody or antigen-binding fragment, or variant, fusion or derivative thereof, does not comprise all of the amino acid sequences of SEQ ID NOS: 1 to 6.

Suitable antibodies, antigen-binding fragments, variants, fusions and derivatives are described above in relation to the uses and methods of the invention. Such antibodies, antigen-binding fragments, variants, fusions and derivatives capable of competing for binding to an integrin α11 subunit with an scFv molecule having the amino acid sequence of SEQ ID NO: 11 may be identified using techniques well known in the art. For example, phage display antibody libraries may first be screened to identify antibodies which bind to an integrin α11 subunit, which antibodies may then be further tested to identify those which compete with an scFv molecule having the amino acid sequence of SEQ ID NO: 11.

In one embodiment of the antibodies, antigen-binding fragments, variants, fusions and derivatives of the ninth aspect of the invention, the integrin α11 subunit, or a heterodimer thereof, is localised on the surface of a cell (such as a chondrocyte or fibroblast).

It will be appreciated by persons skilled in the art that the antibodies, antigen-binding fragments, variants, fusions and derivatives of the ninth aspect of the invention have utility in the above-described uses and methods of the invention.

By “capable of competing” for binding to an integrin α11 subunit, or a heterodimer thereof, with an scFv molecule having the amino acid sequence of SEQ ID NO: 11, we mean that the antibody or antigen-binding fragment, variant, fusion or derivative thereof, or fusion of a said variant or derivative thereof, is capable of inhibiting or otherwise interfering, at least in part, with the binding of the scFv molecule of SEQ ID NO: 11 to the integrin α11 subunit, or heterodimer thereof. Competitive binding may be determined by methods well known to those skilled in the art, such as ELISA (as described above).

For example, the antibody or antigen-binding fragment, variant, fusion or derivative thereof, or fusion of a said variant or derivative thereof, may be capable of inhibiting the binding of the scFv molecule of SEQ ID NO: 11 to the integrin α11 subunit, or heterodimer thereof by at least 10%, for example at least 20%, 30%, 40%, 50%, 60%, 70%, 80%, 90%, 35% or even by 100%.

In one embodiment, the antibody or antigen-binding fragment, or variant, fusion or derivative thereof, is capable of binding to same epitope as an scFv molecule having the amino acid sequence of SEQ ID NO: 11.

In an alternative embodiment, the antibody or antigen-binding fragment, or variant, fusion or derivative thereof, is capable of binding to an epitope distinct from that to which an scFv molecule having the amino acid sequence of SEQ ID NO: 11 binds.

By “epitope” it is herein intended to mean a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. way which helices or sheets are folded or arranged to give a three-dimensional structure, of an antigen.

In one embodiment of the ninth aspect of the invention, the antibody or antigen-binding fragment, or a variant, fusion or derivative thereof, comprises or consists of an intact antibody.

In an alternative embodiment of the ninth aspect of the invention, the antibody or antigen-binding fragment, or a variant, fusion or derivative thereof, comprises or consists of an antigen-binding fragment selected from the group consisting of Fv fragments (e.g. single chain Fv and disulphide-bonded Fv), and Fab-like fragments (e.g. Fab fragments, Fab′ fragments and F(ab)₂ fragments).

It will be appreciated that the antibody may be a recombinant antibody, such as a monoclonal antibody. For example, the antibody or antigen-binding fragment thereof may be human or humanised.

In a further embodiment of the ninth aspect of the invention, the antibody or antigen-binding fragment, or a variant, fusion or derivative thereof is capable of modulating (e.g. inhibiting) the degradation of collagen, in vitro and/or in vivo

The antibodies, antigen-binding fragments, variants, fusions and derivatives of the ninth aspect of the invention may have utility in the treatment of an inflammatory condition, for example selected from the group consisting of arthritic diseases (such as rheumatoid arthritis and osteoarthritis), joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis and autoimmune chronic inflammatory diseases.

In an additional embodiment of the ninth aspect of the invention, the antibody or antigen-binding fragment, or a variant, fusion or derivative thereof further comprises a therapeutic and/or detectable moiety (as described above).

A tenth aspect of the invention provides a nucleic acid molecule encoding an antibody or antigen-binding fragment, or variant, fusion or derivative thereof according to the ninth aspect of the invention, or a component polypeptide chain thereof.

The isolated nucleic acid molecule is suitable for expressing antibody or antigen-binding fragment, or variant, fusion or derivative thereof, according to the ninth aspect of the invention. By ‘suitable for expressing’ is meant that the nucleic acid molecule is a polynucleotide that may be translated to form the polypeptide, for example RNA, or that the polynucleotide (which is preferably DNA) encoding the polypeptide of the invention is inserted into an expression vector, such as a plasmid, in proper orientation and correct reading frame for expression. The polynucleotide may be linked to the appropriate transcriptional and translational regulatory control nucleotide sequences recognised by any desired host; such controls may be incorporated in the expression vector.

Thus, the nucleic acid molecule may be DNA or RNA.

The nucleic acid molecule (or polynucleotide) may be expressed in a suitable host to produce the polypeptide product of the invention. Thus, the polynucleotide encoding the antibody or antigen-binding fragment, or variant, fusion or derivative thereof, may be used in accordance with known techniques, appropriately modified in view of the teachings contained herein, to construct an expression vector, which is then used to transform an appropriate host cell for the expression and production of the polypeptide of the invention (for example, see Sambrook & Russell, 2000, Molecular Cloning, A Laboratory Manual, Third Edition, Cold Spring Harbor, N.Y., which is incorporated herein by reference).

The nucleic acid molecule encoding the polypeptide may be joined to a wide variety of other polynucleotide sequences for introduction into an appropriate host. The companion polynucleotide will depend upon the nature of the host, the manner of the introduction of the polynucleotide into the host, and whether episomal maintenance or integration is desired.

In brief, expression vectors may be constructed comprising a nucleic acid molecule which is capable, in an appropriate host, of expressing the polypeptide binding moiety or compound encoded by the nucleic acid molecule.

A variety of methods have been developed to operably link nucleic acid molecules, especially DNA, to vectors, for example, via complementary cohesive termini. For instance, complementary homopolymer tracts can be added to the DNA segment to be inserted into the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, e.g. generated by endonuclease restriction digestion, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities. The combination of these activities therefore generates blunt-ended DNA segments. The blunt-ended segments are then incubated with a larger molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc., New Haven, Conn., USA.

A desirable way to modify the DNA encoding the polypeptide of the invention is to use PCR. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention or binding moiety thereof. Thus, the DNA encoding the polypeptide may be used in accordance with known techniques, appropriately modified in view of the teachings contained herein, to construct an expression vector, which is then used to transform an appropriate host cell for the expression and production of the compound of the invention or binding moiety thereof. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker, all of which are incorporated herein by reference.

The DNA (or in the case or retroviral vectors, RNA) encoding the polypeptide binding moiety for use in the methods of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the expression vector of the invention are then cultured for a sufficient time and under appropriate conditions known to those skilled in the art in view of the teachings disclosed herein to permit the expression of the polypeptide, which can then be recovered.

Many expression systems are known, including bacteria (for example, E. coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (Yips) and incorporate the yeast selectable markers HIS3, TRP1, LEU2 and URA3. Plasmids pRS413-416 are Yeast Centromere plasmids (Ycps).

Other vectors and expression systems are well known in the art for use with a variety of host cells.

The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No. ATCC 31343). Exemplary eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA. Exemplary mammalian host cells include Chinese hamster ovary (CHO) cells available from the ATCC as CRL 1658 and 293 cells which are human embryonic kidney cells. Exemplary insect cells are SIP cells which can be transfected with baculovirus expression vectors.

Transformation of appropriate cell hosts with a DNA construct of the present invention is accomplished by well-known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y., all of which are incorporated herein by reference. The method of Beggs (1978) Nature 275, 104-109, which is incorporated herein by reference, is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cells, bacterial cells, insect cells and vertebrate cells.

For example, many bacterial species may be transformed by the methods described in Luchansky et al (1988) Mol. Microbiol. 2, 637-646, which is incorporated herein by reference. The greatest number of transformants is consistently recovered following electroporation of the DNA-cell mixture suspended in 2.5 PEB using 6250V per cm at 25 μFD.

Methods for transformation of yeast by electroporation are disclosed in Becker & Guarente (1990) Methods Enzymol. 194, 182, which is incorporated herein by reference.

Successfully transformed cells, i.e. cells that contain a DNA construct of the present invention, can be identified by well-known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208, which is incorporated herein by reference. Alternatively, the presence of the protein in the supernatant can be detected using antibodies as described below.

In addition to directly assaying for the presence of recombinant DNA, successful transformation can be confirmed by well-known immunological methods when the recombinant DNA is capable of directing the expression of the protein. For example, cells successfully transformed with an expression vector produce proteins displaying appropriate antigenicity.

Samples of cells suspected of being transformed are harvested and assayed for the protein using suitable antibodies.

The host cell may be a host cell within a non-human animal body. Thus, transgenic non-human animals which express a polypeptide according to the invention by virtue of the presence of the transgene are included. In one embodiment, the transgenic non-human animal is a rodent such as a mouse. Transgenic non-human animals can be made using methods well known in the art.

Methods of cultivating host cells and isolating recombinant proteins are well known in the art. It will be appreciated that, depending on the host cell, the compounds of the invention (or binding moieties thereof) produced may differ. For example, certain host cells, such as yeast or bacterial cells, either do not have, or have different, post-translational modification systems which may result in the production of forms of compounds of the invention (or binding moieties thereof) which may be post-translationally modified in a different way.

In one embodiment, the host cell is a bacterial cell. Alternatively, the host cell may be a mammalian cell, for example a human cell, for example a cell from the order of primates, such as a human cell or an ape cell, or a cell from the order of rodentia, such as a rat cell, a mouse cell, a hamster cell, a rabbit cell, a squirrel cell, a guinea pig cell, a marmot cell, or a beaver cell.

Further embodiments are where the host cell is a CHO (Chinese hamster ovary) cell, or a HEK (human embryonic kidney) cell.

Thus, an eleventh aspect of the invention provides a vector comprising a nucleic acid molecule according to the tenth aspect of the invention.

Likewise, twelfth aspect of the invention provides recombinant host cell comprising a nucleic acid molecule according to the tenth aspect of the invention or a vector according to the eleventh aspect of the invention.

A thirteenth aspect of the invention provides a method for producing an antibody or antigen-binding fragment, or variant, fusion or derivative thereof, according to the ninth aspect of the invention, the method comprising culturing a host cell according to the twelfth aspect of the invention under conditions which permit expression of the encoded antibody or antigen-binding fragment thereof. It will be appreciated that such methods may also be used to produce an antibody or antigen-binding fragment, or variant, fusion or derivative thereof for use in the methods of the invention.

Such polypeptides can be produced in vitro using a commercially available in vitro translation system, such as rabbit reticulocyte lysate or wheatgerm lysate (available from Promega). For example, the translation system may be rabbit reticulocyte lysate. Conveniently, the translation system may be coupled to a transcription system, such as the TNT transcription-translation system (Promega). This system allows the production of a suitable mRNA transcript from an encoding DNA polynucleotide in the same reaction as the translation.

Persons skilled in the art will further appreciate that the medicaments and agents described above have utility in both the medical and veterinary fields. Thus, the medicaments and agents may be used in the treatment of both human and non-human animals (such as horses, dogs, mice, rats, apes, monkeys, pigs, and cats). Preferably, however, the patient is human.

A fourteenth aspect of the invention provides a pharmaceutical composition comprising an antibody or antigen-binding fragment, or variant, fusion or derivative thereof, according to the ninth aspect of the invention, and a pharmaceutically acceptable excipient, diluent or carrier.

As used herein, ‘pharmaceutical composition’ means a therapeutically effective formulation.

A ‘therapeutically effective amount’, or ‘effective amount’, or ‘therapeutically effective’, as used herein, refers to that amount which provides a therapeutic effect for a given condition and administration regimen (for example, an amount sufficient to inhibit the degradation of collagen). This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

It will be appreciated by persons skilled in the art that such an effective amount of the antibody or antigen-binding fragment, or a variant, fusion or derivative therefor formulation thereof may be delivered as a single bolus dose (i.e. acute administration) or, more preferably, as a series of doses over time (i.e. chronic administration).

It will be further appreciated by persons skilled in the art that the antibody or antigen-binding fragment, or a variant, fusion or derivative thereof, for use in to the methods of the invention may be administered in combination with one or more other conventional agents for the treatment of inflammatory conditions.

For example, in the case of rheumatoid arthritis, suitable conventional agents include, but are not limited to, disease-modifying antirheumatic drugs (DMARDS, e.g. methotrexate), gold salts, antimalarials, sulphasalazine, tetracyclines, cyclosporine, NSAIDs, corticosteroids, Leflunomide (Arava; Aventis), tumour-necrosis factor-α(TNFα) inhibitors, such as etanercept (Enbrel; Amgen), infliximab (Remicade; J&J/Centocor) and adalimumab (Humira; Abbott).

In the case of OA, suitable conventional agents include, but are not limited to analgesics or anti-inflammatory agents, such as acetaminophen (also known as paracetamol), or non-steroidal anti-inflammatory drugs (NSAIDs) inhibiting cyklooxygenase (COX), such as COX2 inhibitors, Disease-Modifying Osteoarthritis Drugs (DMOADs), such as MMP inhibitors, or interleukin 1 (IL-1) inhibitor.

The antibody or antigen-binding fragment, or variant, fusion or derivative thereof, according to the ninth aspect of the invention may be formulated at various concentrations, depending on the efficacy/toxicity of the compound being used. In one embodiment, the formulation comprises the agent of the invention at a concentration of between 0.1 μM and 1 mM, for example between 1 μM and 100 μM, between 5 μM and 50 μM, between 10 μM and 50 μM, between 20 μM and 40 μM or about 30 μM. For in vitro applications, formulations may comprise a lower concentration of a compound of the invention, for example between 0.0025 μM and 1 μM.

Thus, there is provided a pharmaceutical formulation comprising an amount of an antibody or antigen-binding fragment, or variant, fusion or derivative thereof, effective to treat an inflammatory condition (as described above).

It will be appreciated by persons skilled in the art that the medicaments and agents will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA, which is incorporated herein by reference).

For example, the medicaments and agents can be administered orally, buccally or sublingually in the form of tablets, capsules, ovules, elixirs, solutions or suspensions, which may contain flavouring or colouring agents, for immediate-, delayed- or controlled-release applications. The medicaments and agents may also be administered via intracavemosal injection.

Such tablets may contain excipients such as microcrystalline cellulose, lactose, sodium citrate, calcium carbonate, dibasic calcium phosphate and glycine, disintegrants such as starch (for example, corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Exemplary excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The medicaments and agents can also be administered parenterally, for example, intravenously, intra-articularly, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (for example, to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the medicaments and agents will usually be from 1 to 1000 mg per adult (i.e. from about 0.015 to 15 mg/kg), administered in single or divided doses.

It is appreciated that for the prevention or treatment of disease, the appropriate dosage of an antibody will depend on the type of disease to be treated, the severity and of course of the disease, whether the antibody or a fragment thereof is administered for preventative or therapeutic purposes, the course of previous therapy and the patient's clinical history and response to the antibody or a fragment thereof. The antibody, antigen-binding fragment, variant, fusion or derivative thereof according to the present invention is suitably administered to the patient at one time or over a series of treatments. Depending on the type and severity of the disease, about 0.015 to 15 mg of antibody or a fragment thereof/kg of patient weight is an initial candidate dosage for administration to the patient. Administration may be, for example, by one or more separate administrations, or by continuous infusion. For repeated administrations over several days or longer, depending on the condition, the treatment is repeated until a desired suppression or alleviation of the disease symptoms occurs. However, other dosage regimens may be useful and are not excluded.

The effectiveness of antibody, antigen binding fragment, variant, fusion or derivative thereof in alleviating the symptoms, preventing or treating disease may be improved by serial administering or administration in combination with another agent that is effective for the same clinical indication, such as another antibody or a fragment thereof directed against a different epitope than that of the antibody according to the invention, or one or more conventional therapeutic agents known for the intended therapeutic indication,

Suitable pharmaceutically acceptable agents affecting such indications may be anti-inflammatory drugs such as non steroidal anti-inflammatory drugs (NSAIDS) for the treatment of inflammatory diseases such as arthritic diseases e.g. osteoarthritis, rheumatoid arthritis; anti-cytoltine agents e.g. anti-TNF antibodies, interleukin receptor antagonist, matrix metalloproteinase (MMP) inhibitors or bone morphogenic proteins (BMP); local anaesthetics for use post-operatively following orthopaedic surgery for the treatment of pain management or hypolipidemic drugs for treatment of atherosclerotic plaque, matrix metalloproteinase (MMPs) inhibitors or bone morphogenic proteins (BMPs).

The medicaments and agents can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations may be arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the medicaments and agents can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route.

For application topically to the skin, the medicaments and agents can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Where the medicament or agent is a polypeptide, it may be preferable to use a sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Sustained-release immunoglobulin compositions also include liposomally entrapped immunoglobulin. Liposomes containing the immunoglobulin are prepared by methods known per se. See, for example Epstein et al., Proc. Natl. Acad. Sci. USA 82: 3688-92 (1985); Hwang et al., Proc. Natl. Acad. Sci. USA 77: 4030-4 (1980); U.S. Pat. Nos. 4,485,045; 4,544,545; 6,139,869; and 6,027,726, which are incorporated herein by reference. Ordinarily, the liposomes are of the small (about 200 to about 800 Angstroms), unilamellar type in which the lipid content is greater than about 30 mole percent (mol. %) cholesterol; the selected proportion being adjusted for the optimal immunoglobulin therapy.

Alternatively, polypeptide medicaments and agents can be administered by a surgically implanted device that releases the drug directly to the required site.

Electroporation therapy (EPT) systems can also be employed for the administration of proteins and polypeptides. A device which delivers a pulsed electric field to cells increases the permeability of the cell membranes to the drug, resulting in a significant enhancement of intracellular drug delivery.

Proteins and polypeptides can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skirt. The particles can be loaded or coated with drugs or genes or can simply act as “bullets” that generate pores in the skin through which the drugs can enter.

An alternative method of protein and polypeptide delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and polypeptide pharmaceuticals can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant specificity for intrinsic factor (IF) in the vitamin B12 portion of the complex and significant bioactivity of the drug portion of the complex.

The antibody and antibody-derived binding agents according to the ninth aspect of the invention may be provided in the form of a kit comprising a pharmaceutical composition as described above. Thus, a kit may be provided for use in the treatment of an inflammatory condition

Alternatively, the kit may comprise a detectable antibody or antigen-binding fragment or derivative thereof according to the invention, suitable for use in diagnosis. Such a diagnostic kit may comprise, in an amount sufficient for at least one assay, the diagnostic agent as a separately packaged reagent. Instructions for use of the packaged reagent are also typically included. Such instructions typically include a tangible expression describing reagent concentrations and/or at least one assay method parameter such as the relative amounts of reagent and sample to be mixed, maintenance time periods for reagent/sample admixtures, temperature, buffer conditions and the like.

A further aspect of the present invention provides a method for identifying a candidate compound for treating an inflammatory condition, the method comprising the following steps:
a) providing a compound to be tested;
b) testing the ability of the compound to compete for binding to an integrin α11 subunit, or a heterodimer thereof, with an antibody or antigen-binding fragment comprising the following CDR sequences:

| | |
|---|---|
| FSRYYMHWVRQVPG; | [SEQ ID NO: 1] |
| SGVSWNGSRTHYADSVKGR; | [SEQ ID NO: 2] |
| ARVSGDGYNFGA; | [SEQ ID NO: 3] |
| CTGSSSNIGAGYDVH; | [SEQ ID NO: 4] |
| GYNERPS; | [SEQ ID NO: 5] |
| and | |
| CAAWDDSLSGHVV. | [SEQ ID NO: 6] |

or a variant, fusion or derivative thereof, or a fusion of a said variant or derivative thereof, which retains the binding specificity for the integrin α11 subunit, or heterodimer thereof,
   wherein the test compound is identified as a candidate compound for treating an inflammatory condition if it is able to compete for binding to the integrin all subunit, or heterodimer thereof.

Competitive binding may be assayed using methods well known in the art, such as ELISA (see above).

In one embodiment, the method further comprises the step of testing the ability of the compound to modulate (e.g. inhibit) the degradation of collagen.

By “modulate” we include increasing or decreasing the rate of degradation of collagen. It will be appreciated that inhibition of the degradation of collagen by be in whole or in part. For example, collagen degradation may be inhibited by 10% or more, e.g. 20%, 30%, 40%, 50%, 60%, 70%, 80%, 90% or 100%, compared to the degradation of collagen in the absence of the test compound.

Conveniently, some or all of the testing steps are performed in vivo.

Alternatively, some or all of the testing steps may be performed in vitro, for example using immunohistochemical methods (e.g. staining with Safranin) (see Examples below).

Exemplary test compounds include polypeptides, or a fusion or derivative thereof, or a fusion of a said derivative thereof. For example, the test compound may be an antibody of antigen-binding fragment thereof, or a variant, fusion or derivative thereof, or a fusion of a said variant or derivative thereof.

In one embodiment, the test compound is an intact antibody.

Alternatively, the test compound may be an antigen-binding fragment selected from the group consisting of Fv fragments (e.g. single chain Fv and disulphide-bonded Fv), Fab-like fragments (e.g. Fab fragments, Fab′ fragments and F(ab)₂ fragments), single antibody chains (e.g. heavy or light chains), single variable domains (e.g. V_{H} and V_{L} domains) and domain antibodies (dAbs, including single and dual formats [i.e. dAb-linker-dAb]).

A further aspect of the invention provides an epitope defined by an antibody or an antigen-binding fragment thereof with binding specificity for an integrin α11 subunit, or a heterodimer thereof, or of a variant, fusion or derivative of said antibody or an antigen-binding fragment, or a fusion of a said variant or derivative thereof, which retains the binding specificity of said antibody or an antigen-binding fragment thereof for an integrin α11 subunit, or a heterodimer thereof, wherein the antibody or fragment comprises the following amino acid sequences:

| | |
|---|---|
| FSRYYMHWVRQVPG; | [SEQ ID NO: 1] |
| SGVSWNGSRTHYADSVKGR; | [SEQ ID NO: 2] |
| ARVSGDGYNFGA; | [SEQ ID NO: 3] |
| CTGSSSNIGAGYDVH; | [SEQ ID NO: 4] |
| GYNERPS; | [SEQ ID NO: 5] |
| and | |
| CAAWDDSLSGHVV. | [SEQ ID NO: 6] |

In one embodiment, the epitope comprises or consists of the following amino acid sequence:

[SEQ ID NO: 15]
SDGSLECVNEERRLQKQVCNVSYPFFRAKAKVAFRLDFEFSKSIFLHHLE
IELAAGSDSNERDSTKEDNVAPLRFHLKYEADVLFTRSSSLSHYEVKLN
SSLERYDGIGPPFSCIFRIQNLGLFPIHGMMMKITIPIATRSGNRLLKLR
DFLTDEANTSCNIWGNSTEYRPTPVEEDLRRAPQLNHSNSDVVSINCNIR
LVPNQEINFHLLGNLWLRSLKALKYKSMKIMVNAALQRQFHSPFIFREED
PSRQIVFEISKQEDWQVP

or a fragments, fusions or variants thereof.

Exemplary variants, fusions and fragments thereof are disclosed above.

Thus, as an additional aspect, the invention also provides an isolated polypeptide comprising or consisting of the following amino acid sequence:

[SEQ ID NO: 15]
SDGSIECVNEERRLQKQVCNVSYPFFRAKAKVAFRLDFEFSKSIFLHHLE
IELAAGSDSNERDSTKEDNVAPLRFHLKYEADVLFTRSSSLSHYEVKLN
SSLERYDGIGPPFSCIFRIQNLGLFPIHGMMMKITIPIATRSGNRLLKLR
DFLTDEANTSCNIWGNSTEYRPTPVEEDLRRAPQLNHSNSDVVSINCNIR
LVPNQEINFHLLGNLWLRSLKALKYKSMKIMVNAALQRQFHSPFIFREED
PSRQIVFEISKQEDWQVP

or a fragments, fusions or variants thereof.

Said polypeptide may stimulate an immune response and thus work as an immunogenic epitope.

In one embodiment, the polypeptide, fragment, fusion or variant thereof is capable of binding to an antibody or an antigen-binding fragment thereof with binding specificity for an integrin α11 subunit, or a heterodimer thereof, or of a variant, fusion or derivative of said antibody or an antigen-binding fragment, or a fusion of a said variant or derivative thereof which retains the binding specificity of said antibody or an antigen-binding fragment thereof for an integrin α11 subunit, or a heterodimer thereof,

wherein the antibody or fragment comprises the following amino acid sequences:

| | |
|---|---|
| FSRYYMHWVRQVPG; | [SEQ ID NO: 1] |
| SGVSWNGSRTHYADSVKGR; | [SEQ ID NO: 2] |
| ARVSGDGYNFGA; | [SEQ ID NO: 3] |
| CTGSSSNIGAGYDVH; | [SEQ ID NO: 4] |
| GYNERPS; | [SEQ ID NO: 5] |
| and | |
| CAAWDDSLSGHVV. | [SEQ ID NO: 6] |

An additional aspect of the invention provides the use of the above-described epitopes and isolated polypeptides to identify or produce an antibody or antigen-binding fragment, or variant, fusion or derivative thereof, according to the ninth aspect of the invention.

The present invention also encompasses isolated nucleic acid molecules encoding the above-described epitopes and isolated polypeptides, as well as vectors, host cells and methods of making the same.

As used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

Non-limiting examples which embody certain aspects of the invention will now be described, with reference to the following figures:

### BRIEF DESCRIPTION OF THE FIGURES

FIG. 1: Screening the n-CoDeR® antibody library for anti-alpha 11 integrin binders. One hundred and ninety two 192 screened alpha 11 integrin active scFv clones were confirmed for binding to α11 integrin transfected 293 cells, but no binding to α10 integrin transfected 293 cells, by co-incubation of E. coli expressed His-tagged scFv clones with integrin transfected cells (as indicated in figure), mouse anti-His mAb, Cy5-conjugated anti-mouse mAb reagent, and subsequent screening in an FMAT macroconfocal screening instrument. Screened scFv were retrieved following panning of the n-CoDeR® antibody library for α11 integrin specific binders. One hundred and seventy two 172 scFv clones were verified as selective α11 integrin binders using this approach.

FIG. 2: Target integrin specificity of anti-integrin scFv. Hek 293 cells transfected with human α10 integrin (left graph) or human α11 integrin (right graph) were incubated with anti-α10 integrin scFv (control, grey peak with label), anti-α11 integrin scFv (grey peak with label) or irrelevant control scFv (solid black peak). Binding of scFv to cells was analysed by flow-cytometry following detection with secondary Phycoerythrin conjugated anti-His mAb.

FIG. 3: The ability of anti-alpha11 integrin scFv to selectively inhibit adhesion of alpha11beta1 integrin transfected C2C12 cells to collagen II coated plates. Alpha 10 integrin transfected C2C12 mouse fibroblast cells (A) or α11 integrin transfected C2C12 mouse fibroblast cells (B) were pre-incubated with 10 μg/ml of anti-α11 integrin scFv, FITC-8 control scFV; anti-α10 integrin monoclonal antibody, or IgG2a isotype control antibody. Cells were then added to collagen II coated microliter plates and were allowed to adhere. Unbound cells were washed away and the number of bound cells determined by an enzymatic assay. Graph represent combined data from three independent experiments and data from each experiment was normalized to the control without antibody. (Top) ScFvs effect on cell adhesion of α10 transfected cells. (Bottom) ScFvs effect on cell adhesion of α11 transfected cells. Mean values with s.e.m. Significance calculated using one-way Anova and Dunnett's multiple comparison test. White bars: α11-scFvs. Striped bars: controls.

FIG. 4: Lack of binding of n-CoDeR® anti-α11 integrin scFv to peripheral blood leukocyte subpopulations. Purified human PBLs were incubated with anti-α11 integrin scFv α11_A_{—}001-A03-MH (grey line), positive control scFv C11 (black dotted line) or negative control scFv FITC-8 (solid black peak) in the presence of differently fluorochrome conjugated cell subpopulation specific monoclonal antibodies. Cells were washed and incubated with 1/20 PE-conjugated anti-His antibodies (R&D Systems). Positive binding of anti-α11β1 integrin specific scFv was recorded as FL2 fluorescence intensities greater than those observed for 97-99% of cells stained with negative control CT17 scFv. Prior to incubation with mouse anti-His antibodies, cells were pre-blocked and kept in the presence of 200 μg/ml of mouse IgG per 2×10⁶ total leukocytes T cells, B cells, and NK cells were defined as CD3+, CD19+, CD3−CD56+ cells (gates 5, 4 and 3 respectively in left hand graphs). Monocytes (R2) and granulocytes (R1) were defined by unique forward scatter and side scatter properties and additionally as being CD3−CD19−CD56−.

FIG. 5: Ratio of joint swelling, measured by Tc-99m uptake, in induced and non-induced knee at day 3 after i.a. injection of mBSA. Key: ‘CT17’=Control Ab (to cholera toxin), ‘A03’=exemplary anti-alpha11 whole IgG antibody.

FIG. 6: Histological score of inflammation as infiltrating cells in synovial space in sections of whole knee joint. Arbitrary scale from 0 to 3: 0—no inflammation, 1—minor inflammation, 2—moderate inflammation, 3—severe inflammation. Key: ‘CT17’=Control Ab, ‘A03’=exemplary anti-alpha11 antibody.

FIG. 7: Histological score of exudate, infiltrating of cells the joint space, in sections of whole knee joint. Arbitrary scale from 0 to 3: 0—no inflammation, 1—minor inflammation, 2—moderate inflammation, 3—severe inflammation. Key: ‘CT17’=Control Ab, ‘A03’=exemplary anti-alpha11 antibody.

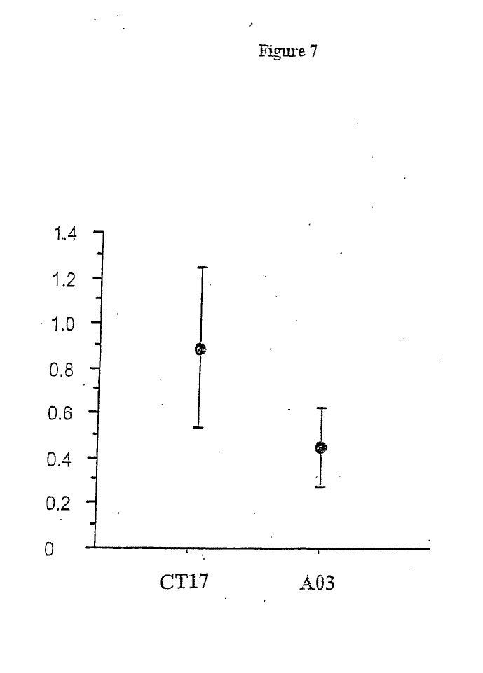

FIG. 8: Proteoglycan (PG) depletion on day 15 after arthritis induction in patella cartilage measured by image analysis. Key: ‘CT17’=Control Ab, ‘A03’=exemplary anti-alpha11 antibody.

FIG. 9: PG depletion—A03 reduces PG depletion on day 15 after arthritis induction in first experiment; FIG. 11A shows histological scoring from patella (shown as scoring by image analysis in FIG. 10), FIG. 11B shows histological scoring of the lateral tibia, and FIG. 11C shows an overview of the knee.

FIG. 10: PG depletion—A03 reduces PG depletion on day 15 after arthritis induction in second experiment. FIG. 12A shows histological scoring from patella, FIG. 12B shows histological scoring of the lateral tibia, (p=0.02 for lateral tibia)

FIG. 11: A03 reduces osteophyte size as measured on day 15 after arthritis induction.

FIG. 12: Incidence (frequency) of arthritis in G6PI-induced arthritis. Treatment was administrated day 7, 10, 13 and 16 after G6PI immunization.

FIG. 13: Mean clinical score of arthritis in G6PI-induced arthritis. Treatment was administrated day 7, 10, 13 and 16 after G6PI immunization.

FIG. 14: Weight change (in %) at day 13 after G6PI immunization. Mice treated with control antibody lost mean weight of 10%. A03 treated mice lost at mean 5% in weight, clear reduction in weight loss compared to control group (p=0.08).

### EXAMPLES

### Example I

### Production of Exemplary Antibody of the Invention (Designated ‘A03’

### Identification of N-CoDeR®-Derived Human Antibodies Specific for α11β1 Integrin

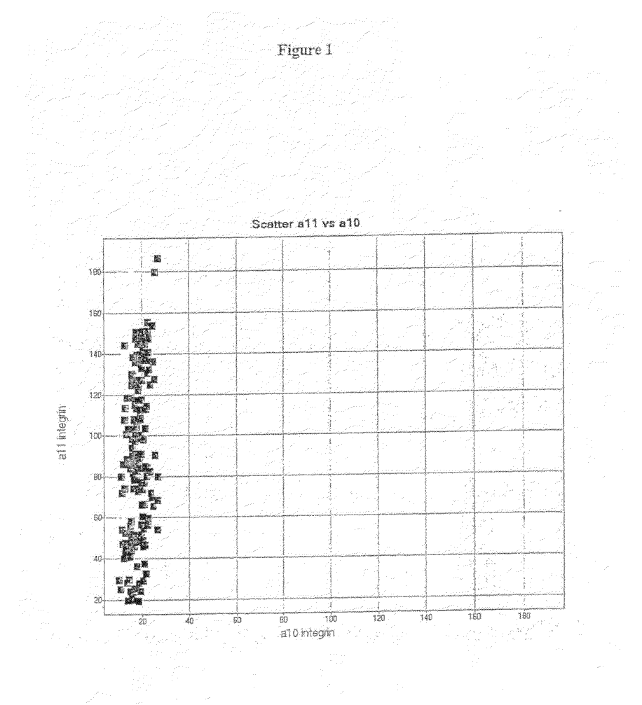

The fully human antibody library n-CoDeR® (see Soderlind et al., 2000, Nat Biotechnol 18:852-6 and WO 98/32845, which are incorporated herein by reference) was screened for antibody fragments (scFv) with specificity for the α10β31 integrin or the α11β1 integrin. A series of combined positive and subtractive panning methodologies was devised in order to maximise retrieval of highly target integrin specific antibodies (see Appendix below). Following conversion to scFv format, antibody fragments were expressed in E. coli and were positively or negatively screened for binding to Hek-293 cells expressing α10β1 integrin or α11β1 integrin, as appropriate, using FMAT technology (FIG. 1, FMAT scatter plot). Thus, hundreds of genotype unique antibody fragments specific for α10β1 integrin or α11β1 integrin were identified (Table 1).

### Antibody Panning Procedure

A phage stock of n-CoDeR® scFv library in a buffer of 3% BSA, 0.02% sodium azide, 0.1% NP40, 10 mM MgCl₂ and 0.01 mM CaCl₂ in PBS was pre-selected over night at 4° C. using BSA coated in an immunotube and tosyl activated Dynabeads M-280 (Dynal Cat. #142.04) coupled with rabbit-anti-alfa10 integrin and rabbit-anti-alfa11 integrin polyclonal antibodies according to Dynals' instructions. The first panning was performed over night at 4° C. on lysate from 15×10⁶ HEK293 cells expressing α₁₁β₁ loaded on magnetic beads coupled with rabbit-anti-alfa11 polyclonal antibody (as above). Beads were washed with 9×1 ml buffer and bound phage were eluted with trypsin and amplified as described elsewhere (Hallborn & Carlsson, 2002, Biotechniques December; Suppl:30-37).

Amplified phage from the 1^{st} panning, buffered as above, were pre-selected on polyclonal coupled Dynabeads as in 1^{st} panning followed by a selection analogous to the first panning with lysate from 10×10⁶ HEK293 α₁₁β₁ cells loaded on the Dynabeads with rabbit-anti-alfa11 polyclonal antibody. After washing (9×1 ml buffer) bound phage were eluted with trypsin and amplified.

Amplified phage from the 2^{nd} panning, diluted in DMEM cell medium containing 10% FCS, 10 mM MgCl₂ and 10 μM CaCl₂, were pre-selected over night at 4° C. against 45×10⁶ C2C12 cells expressing α₁₀β₁ and 13×10⁶ HEK cells expressing α₁₀β₁. Phages were then incubated for 4 h at 4° C. with α₁₁β₁ displayed on recombinant C2C12 cells (5×10⁶ cells used). After density centrifugation through 40% Ficoll (containing 2% FCS, 10 mM MgCl₂ and 10 μM CaCl₂) to remove unbound phages, remaining binders were eluted with both 76 mM citric acid pH 2.5 and 200 mM triethanolamine. The pool of eluted phages were amplified in Escherichia coli HB101F′ for conversion to scFv format.

Conversion to scFv Format

Conversion to c-myc/6×his scFv format (EagI conversion) Protein III is the functional link between the scFv and the phage particle and removal of gene III results in production of soluble scFv. Phagemid DNA from the n-CoDeR Lib2000 selection is digested with EagI to remove gene III and bring the 6×his tag next to the scFv fragment and c-myc tag. The plasmid is ligated and a “killer-cut” with EcoRI, which has a restriction site within gene III, is done to eliminate re-ligated phagemids. The plasmid DNA is transformed into chemically competent Escherichia coli TOP 10.

TABLE 1
Number of α11 integrin specific antibody fragments with unique
genotype isolated from the n-CoDeR ® library

| | Type of antibody | |
|---|---|---|
| Specificity | fragment | Number of clones |
| Anti α11 integrin | scFv | 60 |
| Anti α11 integrin | Fab | 11 |

Specificity of Antibodies for their Targeted Integrin α-Chain

To date four different collagen binding integrin heterodimers have been identified, namely α1β1, α2β1, α10β1 and α11β1. Their inter-individual preference for binding to different types of collagen is incompletely characterised. Among the collagen binding integrins α10β1 α11β1 integrin and α11β1 integrin show high homology (60% identity at amino acid level of ligand binding I-domains) (see Gullberg et al., 2003, Structure and function of alphalpha11beta1 integrin. In: D. Gullberg (Ed) I Domains in Integrins.). Consequently, some antibodies raised against either of these integrin heterodimers are likely to cross-react with the other.

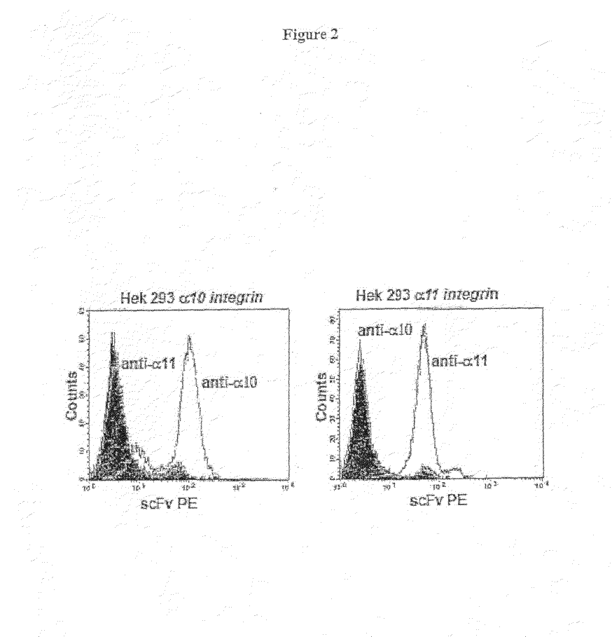

In order to be able to attribute putative observed effects of the isolated anti-α11β1 integrin antibodies on extracellular matrix turnover to exclusive interactions with α11β1 integrin heterodimers, the fine specificity of antibodies for this complex was determined. Thus, anti-α11β1 integrin scFv were examined for binding to α10β1 integrin transfected or α11β1 integrin transfected Hek-293 cells using flow-cytometry. By this approach, twenty-nine anti-α11β1 integrin scFv (out of 38 examined) that bound strongly to α11β1 integrin transfected cells, but not to α10β1 integrin transfected cells were identified (FIG. 2).

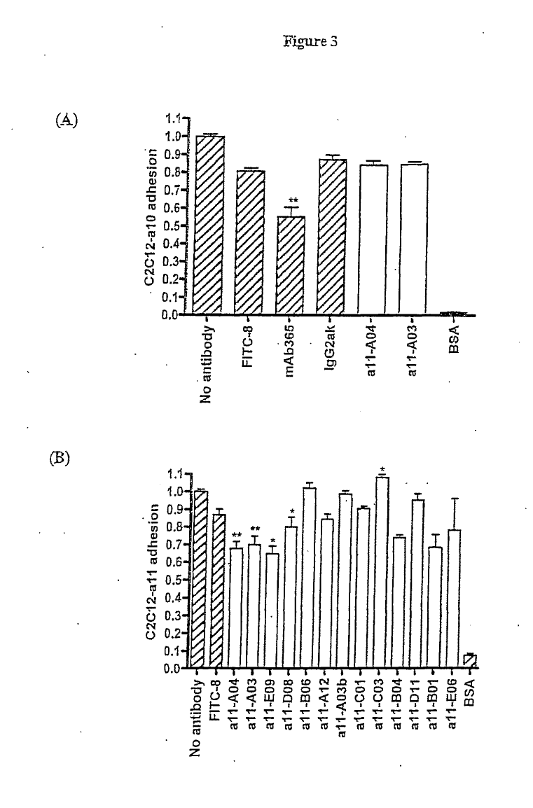

The fine-specificity of anti-integrin antibodies is further illustrated by the ability of anti-α11β1 integrin scFv to selectively inhibit adhesion of α11β1 integrin transfected (but not α10β1 integrin transfected) C2C12 cells to collagen II coated plates (FIG. 3). Considering the integrin structure and the structural changes taking place during integrin activation and binding an agent that interferes with cell adhesion (i.e. integrin binding) is potentially interesting. Antibodies or scFv's that bind at or close to the ligand-binding site (i.e. MIDAS) could physically interfere with ligand interaction. Antibodies or scFv's that do not bind close to MIDAS (e.g. not I-domain binders) but still have an effect on cell adhesion are likely to interfere with integrin alpha chain structure, integrin activation or alpha/beta chain interactions.

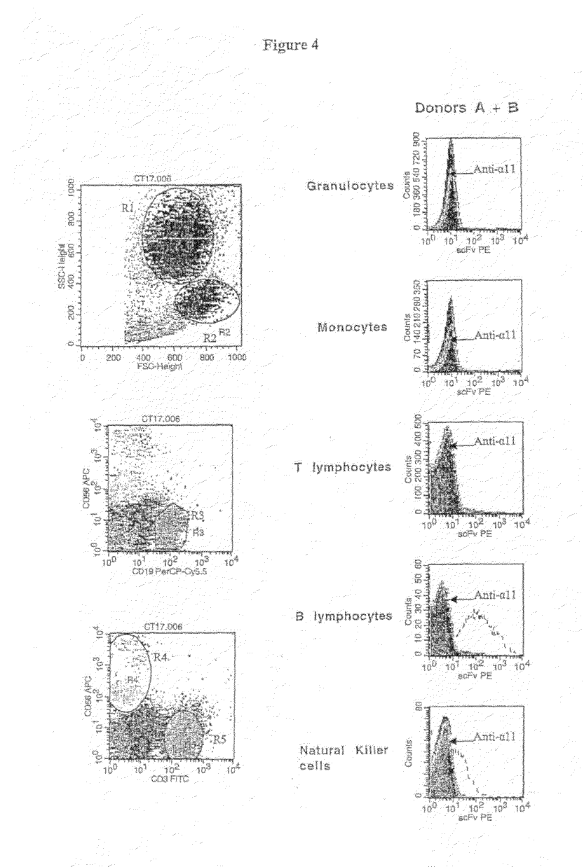

Finally, prior to examination in functional in vitro and in vivo test systems anti-α11β1 integrin antibody clones were shown not to cross-react with defined peripheral blood leukocyte subpopulations including Granulocytes, Monocytes, T lymphocytes, B lymphocytes, and Natural Killer cells (FIG. 4).

### Sequence Information on Exemplary Anti-α11 Antibodies

Heavy chain of anti-all whole IgG antibody
(A03)- amino acid sequence
[SEQ ID NO: 11]
EVQLLESGGGLVQPGGSLRLSCAASGFTFSRYYMHWVRQVPGKGLEW
VSGVSWNGSRTHYADSVKGRFTISRDNSKNTLYLQMNSLRAEDTAVY
YCARVSGDGYNFGAWGQGTLVTVSSASTKGPSVFPLAPCSRSTSESTAA
LGCLVKDYFPEPVTVSWNSGALTSGVHTFPAVLQSSGLYSLSSVVTVPS
SSLGTKTYTCNVDHKPSNTKVDKRVESKYGPPCPSCPAPEFLGGPSVFL
FPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHY
TQKSLSLSLGK
Light chain of anti-all whole IgG antibody
(A03)- amino acid sequence
[SEQ ID NO: 12]
QSVLTQPPSASGTPGQRVTISCTGSSSNIGAGYDVHWYQQLPGTAPKLLI
YGYNERPSGVPDRFSGSKSGTSASLAISGLRSEDEADYYCAAWDDSLSG
HVVFGGGTKLTVLGQPKAAPSVTLFPPSSEELQANKATLVCLISDFYPG
AVTVAWKADSSPVKAGVETTTPSKQSNNKYAASSYLSLTPEQWKSHRS
YSCQVTHEGSTVEKTVAPTECS
ScFv format of anti-all antibody (A03)-
amino acid sequence
[SEQ ID NO: 13]
EVQLLESGGGLVQPGGSLRLSCAASGFTFSRYYMHWVRQVPGKGLEW
VSGVSWNGSRTHYADSVKGRFTISRDNSKNTLYLQMNSLRAEDTAVY
YCARVSGDGYNFGAWGQGTLVTVSSGGGGSGGGGSGGGGSQSVLTQP
PSASGTPGQRVTISCTGSSSNIGAGYDVHWYQQLPGTAPKLLIYGYNER
PSGVPDRFSGSKSGTSASLAISGLRSEDEADYYCAAWDDSLSGHVVFGG
GTKLTVLG
Heavy chain of anti-all whole IgG antibody
(A03)- nucleotide sequence
[SEQ ID NO: 16]
GAGGTGCAGCTGTTGGAGTCTGGGGGAGGCTTGGTACAGCCTGGGG
GGTCCCTGAGACTCTCCTGTGCAGCCTCTGGATTCACCTTCAGTCGTT
ACTACATGCACTGGGTCCGCCAAGTTCCAGGGAAGGGGCTGGAGTG
GGTATCGGGTGTTAGTTGGAATGGCAGTAGGACGCACTATGCAGAC
TCTGTGAAGGGCCGATTCACCATCTCCAGAGACAATTCCAAGAACAC
GCTGTATCTGCAAATGAACAGCCTGAGAGCCGAGGACACTGCCGTG
TATTACTGTGCGAGAGTAAGTGGAGATGGCTACAATTTTGGCGCCTG
GGGCCAGGGTACACTGGTCACCGTGAGCTCAGCTTCCACCAAGGGC
CCATCCGTCTTCCCCCTGGCGCCCTGCTCCAGGAGCACCTCCGAGAG
CACAGCCGCCCTGGGCTGCCTGGTCAAGGACTACTTCCCCGAACCGG
TGACGGTGTCGTGGAACTCAGGCGCCCTGACCAGCGGCGTGCACAC
CTTCCCGGCTGTCCTACAGTCCTCAGGACTCTACTCCCTCAGCAGCG
TGGTGACCGTGCCCTCCAGCAGCTTGGGCACGAAGACCTACACCTGC
AACGTAGATCACAAGCCCAGCAACACCAAGGTGGACAAGAGAGTTG
AGTCCAAATATGGTCCCCCATGCCCATCATGCCCAGCACCTGAGTTC
CTGGGGGGACCATCAGTCTTCCTGTTCCCCCCAAAACCCAAGGACAC
TCTCATGATCTCCCGGACCCCTGAGGTCACGTGCGTGGTGGTGGACG
TGAGCCAGGAAGACCCCGAGGTCCAGTTCAACTGGTACGTGGATGG
CGTGGAGGTGCATAATGCCAAGACAAAGCCGCGGGAGGAGCAGTTC
AACAGCACGTACCGTGTGGTCAGCGTCCTCACCGTCCTGCACCAGGA
CTGGCTGAACGGCAAGGAGTACAAGTGCAAGGTCTCCAACAAAGGC
CTCCCGTCCTCCATCGAGAAAACCATCTCCAAAGCCAAAGGGCAGC
CCCGAGAGCCACAGGTGTACACCCTGCCCCCATCCCAGGAGGAGAT
GACCAAGAACCAGGTCAGCCTGACCTGCCTGGTCAAAGGCTTCTACC
CCAGCGACATCGCCGTGGAGTGGGAGAGCAATGGGCAGCCGGAGAA
CAACTACAAGACCACGCCTCCCGTGCTGGACTCCGACGGCTCCTTCT
TCCTCTACAGCAGGCTAACCGTGGACAAGAGCAGGTGGCAGGAGGG
GAATGTCTTCTCATGCTCCGTGATGCATGAGGCTCTGCACAACCACT
ACACACAGAAGAGCCTCTCCCTGTCTCTGGGTAAA
Light chain of anti-all whole IgG antibody
(A03)- nucleotide sequence
[SEQ ID NO: 17]
CAGTCTGTGCTGACTCAGCCACCCTCAGCGTCTGGGACCCCCGGGCA
GAGGGTCACCATCTCCTGCACTGGGAGCAGCTCCAACATCGGGGCA
GGTTATGATGTACACTGGTATCAGCAGCTCCCAGGAACGGCCCCCAA
ACTCCTCATCTATGGTTATAATGAGCGGCCCTCAGGGGTCCCTGACC
GATTCTCTGGCTCCAAGTCTGGCACCTCAGCCTCCCTGGCCATCAGT
GGGCTCCGGTCCGAGGATGAGGCTGATTATTACTGTGCAGCATGGG
ATGACAGCCTGAGTGGTCATGTGGTATTCGGCGGAGGAACCAAGCT
GACGGTCCTAGGTCAGCCCAAGGCTGCCCCCTCGGTCACTCTGTTCC
CGCCCTCCTCTGAGGAGCTTCAAGCCAACAAGGCCACACTGGTGTGT
CTCATAAGTGACTTCTACCCGGGAGCCGTGACAGTGGCCTGGAAGG
CAGATAGCAGCCCCGTCAAGGCGGGAGTGGAGACCACCACACCCTC
CAAACAAAGCAACAACAAGTACGCGGCCAGCAGCTATCTGAGCCTG
ACGCCTGAGCAGTGGAAGTCCCACAGAAGCTACAGCTGCCAGGTCA
CGCATGAAGGGAGCACCGTGGAGAAGACAGTGGCCCCTACAGAATG
TTCA
ScFv format of anti-all antibody (A03)-
nucleotide sequence
[SEQ ID NO: 18]
GAGGTGCAGCTGTTGGAGTCTGGGGGAGGCTTGGTACAGCCTGGGG
GGTCCCTGAGACTCTCCTGTGCAGCCTCTGGATTCACCTTCAGTCGTT
ACTACATGCACTGGGTCCGCCAAGTTCCAGGGAAGGGGCTGGAGTG
GGTATCGGGTGTTAGTTGGAATGGCAGTAGGACGCACTATGCAGAC
TCTGTGAAGGGCCGATTCACCATCTCCAGAGACAATTCCAAGAACAC
GCTGTATCTGCAAATGAACAGCCTGAGAGCCGAGGACACTGCCGTG
TATTACTGTGCAAGAGTAAGTGGAGATGGCTACAATTTTGGCGCCTG
GGGCCAGGGTACACTGGTCACCGTGAGCAGCGGTGGAGGCGGTTCA
GGCGGAGGTGGATCCGGCGGTGGCGGATCGCAGTCTGTGCTGACTC
AGCCACCCTCAGCGTCTGGGACCCCCGGGCAGAGGGTCACCATCTCC
TGCACTGGGAGCAGCTCCAACATCGGGGCAGGTTATGATGTACACT
GGTATCAGCAGCTCCCAGGAACGGCCCCCAAACTCCTCATCTATGGT
TATAATGAGCGGCCCTCAGGGGTCCCTGACCGATTCTCTGGCTCCAA
GTCTGGCACCTCAGCCTCCCTGGCCATCAGTGGGCTCCGGTCCGAGG
ATGAGGCTGATTATTACTGTGCAGCATGGGATGACAGCCTGAGTGGT
CATGTGGTATTCGGCGGAGGAACCAAGCTGACGGTCCTAGGT

Initial screening experiments were performed using the scFv format of exemplary antibody ‘A03’.

In vivo studies were performed using the whole IgG format of exemplary antibody ‘A03’.

Appendix I.—Selection Strategies Utilized to Isolate Anti-Alpha11Beta1 Integrin scFv

Selection strategies E to G (lower graph) are exemplified by selections performed with the aim to retrieve a11b1 integrin specific antibodies.

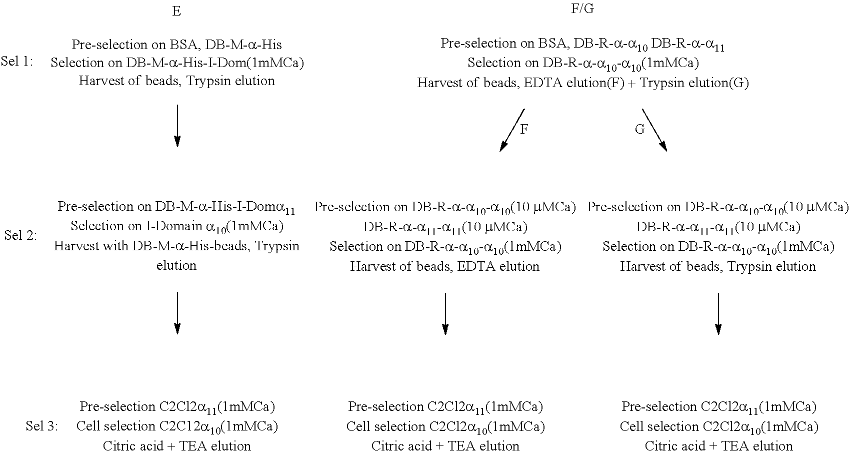

### Example II

### Effect of Exemplary Antibody of the Invention on Antigen-Induced Arthritis

The purpose of the study was to test the effect of an exemplary whole IgG antibody of the invention, designated ‘A03’, in mBSA antigen-induced arthritis (AIA) in C57B1/6 mice.

### Materials and Methods

### Testing Facility

Experimental Rheumatology & Advanced Therapeutics, University Medical Center Nijmegen, Geert Grooteplain 26-28, 6525 GA Nijmegen, The Netherlands.

### Experimental Design

The experimental protocol is shown in Tables 2 and 3 below.

TABLE 2

| Day | Procedure |
|---|---|
| −21 | Immunization of mice with mBSA |
| −14 | Booster with mBSA |
| 0 | Intra-articular injection with mBSA (60 μg/knee) |
| 1, 5, 9, 13 | Administration of anti-integrin antibodies |
| 3, 10 | Measurement of Technetium Uptake |
| 15 | Sacrifice of animals, taking tissues for histology |

TABLE 3

| | | | |
|---|---|---|---|
| | | Target | Dose total |
| Group | Test drug | integrin | (μg/mouse) |

Experiment 1

| | | | |
|---|---|---|---|
| 1 (control antibody) | IgG4 CT17 | — | 900 |
| 2 | IgG4 A03 Batch 1 | alpha 11 | 900 |
| 3 | IgG4 A04 | alpha 11 | 900 |

Experiment 2

| | | | |
|---|---|---|---|
| 1 (control antibody) | IgG4 CT17 | — | 900 |
| 2 (control) | PBS | — | N/A |
| 3 | IgG4 A03 Batch 1 | alpha 11 | 900 |
| 4 | IgG4 A03 Batch 2 | alpha 11 | 900 |

### Animals

Male C57B1/6 mice (Jackson Laboratories, Bar Harbor, Me., USA) of the age of 10 weeks (23-25 g) were used for the study. Mice are acclimatised after arrival for at least one week. Food and acidified water available ad libitum. The mice were weighed weekly.

### Induction of Antigen-Induced Arthritis

Antigen-induced arthritis was induced using a similar protocol as described (1). Shortly, mice were immunized with 100 μg, (microgram) of methylated BSA (mBSA, Sigma) emulsified in Freund's complete adjuvant (CFA) (100 μl mg/ml) on day −21. Injection was divided over both flanks and footpath of the forelegs. Heat-killed Bordetella pertussis (RIVM, Bilthoven, The Netherlands), 1 ml, was administrated i.p. as an additional adjuvant. Seven days after the initial immunization animals were boosted with 100 μg (100 μl mg/ml) mBSA in CFA in the neck and 1 ml Bordetella pertussis (i.p.). Two weeks after the booster injection arthritis was induced by intra-articular injection of 60 μg mBSA (mBSA) in the right knee joint of the mice.

### Antibodies

Antibodies were produced by Bioinvent International AB (Lund, Sweden).

### Administration of Test Antibodies

Antibodies were stored at 4° C. after arrival from Cartela AB (Lund, Sweden) to the test facility. Antibodies were dissolved to obtain the correct concentration in sterile PBS (phosphate-buffered saline) and stored under sterile conditions at 4° C. Administration of the antibodies drugs was started on day 1 and ended on day 13. Antibody administration was performed on day 1, 5, 9 and 13. The antibodies were given i.p. 200 μl/mouse. Administration of the antibodies was always performed at the same time of the day −/+1 hr. A total of 900 μg antibody was give per mouse in the following scheme:

TABLE 4

| | | Microgram antibody per |
|---|---|---|
| Day | Date | mouse |
| 0 | i.a. injection mBSA | |
| 1 | | 300 |
| 5 | | 200 |
| 9 | | 200 |
| 13 | | 200 |

### Scoring Inflammation as Measurement of Isotope Uptake

Joint swelling was measured using ⁹⁹Tc pertechate uptake in the knee joint on day 3 and 10. Briefly, mice were injected i.p. with 12 μCi of ⁹⁹Tc and subsequently sedated with chloradehydrate. Thirty minutes thereafter, gamma-radiation was assessed by use of a collimated Na—I-scintillation crystal with the knee in a fixed position. Arthritis was scored as the ration of the ⁹⁹Tc uptake in the right (injected) knee and the left (non-injected) knee. Ratios>1.1 were taken to indicate inflammation of the right knee joint. This method has previously been shown to correlate well with histological findings (2).

### Histology

Both right and left total knee joints were dissected and processed for histology as previously described (1). Shortly, the knees were fixed in phosphate buffered formalin (pH 7.4) for 3 days, decalcified in 5% buffered formic acid, and subsequently embedded in paraffin wax. Semi serial coronal, 7 μm sections were cut on a microtome. The sections were stained by haematoxylin and eosin (H&E) or by Safranin-O (Safranin O: Merck #1.15948) and Fastgreen (Fast Green FCF: Merck #1.04022). Histological parameters (joint inflammations, proteoglycan depletion, osteophyte formation) were scored by light microscopy. Proteoglycan depletion in patella was also scored using digital image analysis.

### Scoring of Inflammation

Frontal sections of whole knee were stained by H&E. Histological scoring of inflammation was by light microscopy. The degree of inflammation was scored as the amount of infiltrating cells in the synovial space and as the amount of exudate, cells in the joint space. Both exudate and infiltrate were scored using an arbitrary scale from 0 to 3: 0—no inflammation, 1—minor inflammation, 2—moderate inflammation, 3—severe inflammation (1).

### Scoring of Cartilage Damage

Total knee joint sections stained with Safranin-O and Fastgreen. Loss of red staining from 6 positions in various cartilage layers (patella, patella femoris, lateral tibia, lateral femur, medial tibia, medial femur), as a measurement of loss of proteoglycan, was determined using an arbitrary scale from 0 to 3: 0—no proteogycan depletion, 1—minor proteogycan depletion, 2—moderate proteogycan depletion, 3—severe proteogycan depletion (1). Proteoglycan depletion in patella was also scored using digital image analysis.

### Scoring of Osteophytes

The size of the osteophyte was scored on sections from medial femur stained by H&E using an arbitrary scale from 0 to 3: 0—no osteophytes, 1—Signs of chondrogenesis, 2—small osteophytes, 3—large osteophytes.

### Statistics

Cartilage proteoglycan depletion damage was analysed using non-parametric Kruskal-Wallis test and Dunn's post test. Differences between groups were tested with the Mann-Whitney test. All locations were tested separately. The technetium uptake was tested using the Student t-test. To perform the statistical calculations the Graphpad Prism 4 software was used.

### Results

Treatment with Antibody Against α11-Integrin Reduced Inflammation in Arthritic Mice

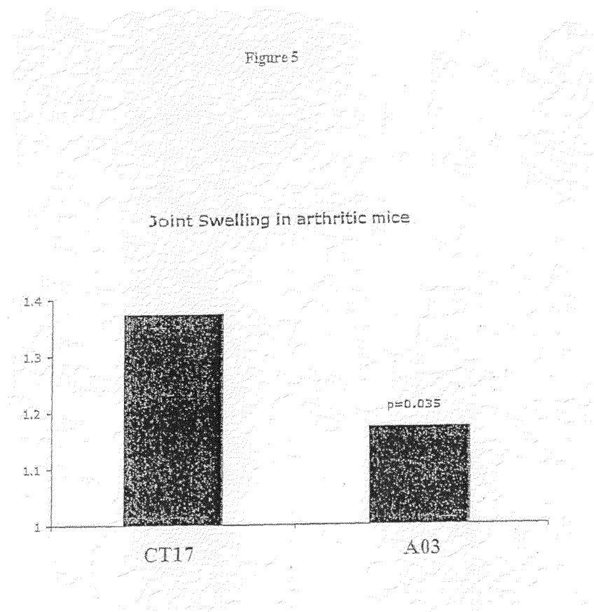

Mono-arthritis was induced by mBSA injection i.a. into the knee joint. The disease has a fast onset where the joint swelling reached its maximum on day 1 after i.a. injection and gradually declining until day 7. The mice were sacrificed after additional 8 days (day 15 after i.a. injection) in the more chronic phase of the disease. The treatment of arthritic mice with anti-α11 integrin antibody (A03) resulted in a significant reduction in inflammation, measure as the degree of swelling of knee joints in the mice as well as degree of infiltrating inflammatory cells into the joint. There was a significant reduction in swelling of the induced knee joint at day 3 after i.a. injection of mBSA in the mice treated with anti-α11 integrin antibody (A03) compared to mice treated with control antibody (CT17) (P=0.035, T test). (FIG. 5). Similar results were shown in two subsequent experiments.

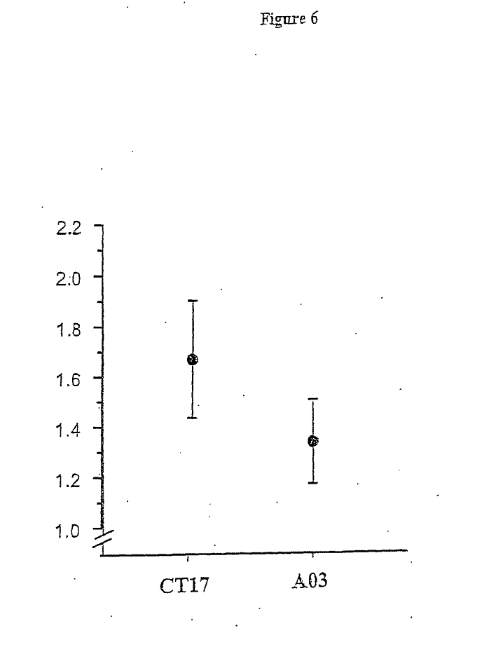

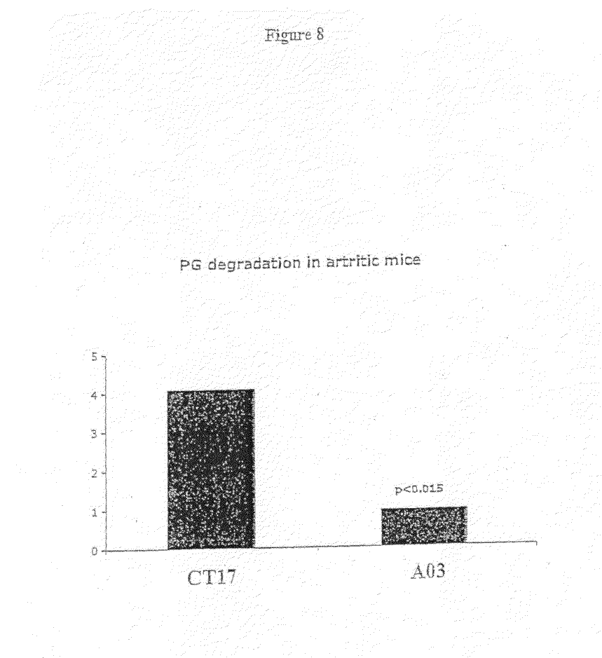

The anti-α11 integrin treated mice had lower amount of infiltrating cells in the synovial space (FIG. 6) and in the joint space (exudate) (FIG. 8) at the end of the experiment (day 15 after i.a. injection) compared to mice treated with control antibody, although the difference was not statistically different.

Treatment with Antibody Against Alfa11 Protects the Cartilage from Destruction in Arthritic Mice

In the mice treated with the anti-all integrin antibody, less destruction of the cartilage was seen compared to the mice treated with control antibody (P=0.015). (FIG. 8). The amount of cartilage destruction was scored as loss of proteoglycans in histological section using an arbitrary scale from 0 to 3 as well as using image analysis. The protective effect of the anti-α11 integrin treatment on proteoglycan depletion was seen at all six locations scored in the knee.

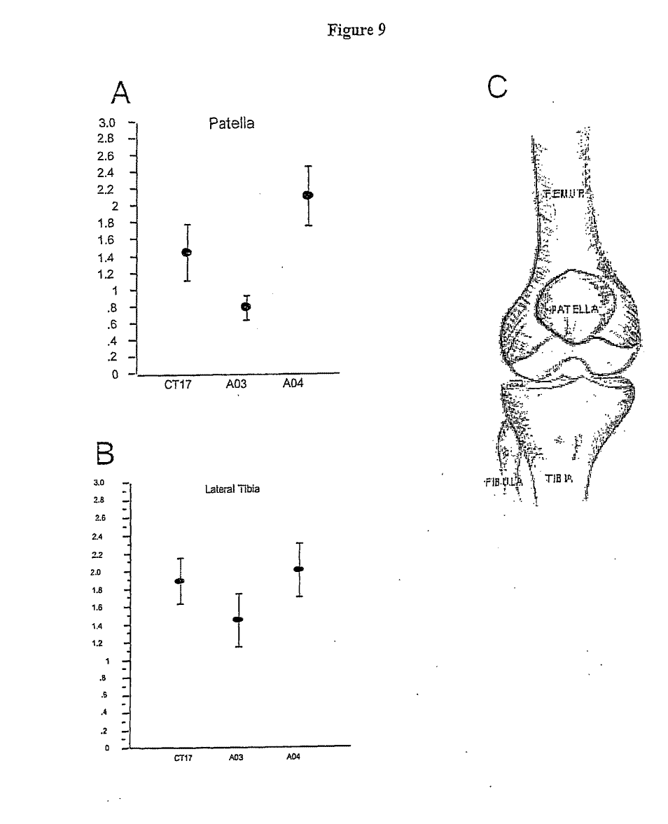

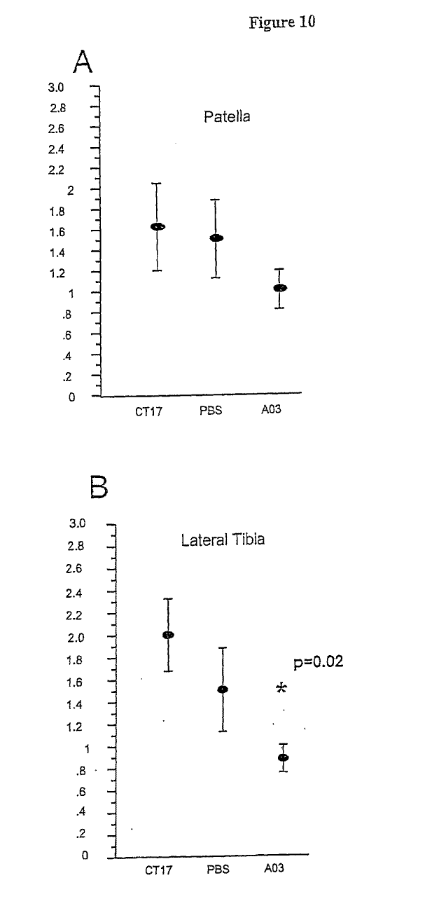

FIGS. 9 and 10 further show the effect of treatment with the anti-all integrin antibody on patella and lateral tibia, measured as proteoglycan (PG-depletion and reduction thereof using an arbitrary scale measurement. As could be seen in the two figures, which represent two different experiments, treatment with A03 reduces the PG-depletion in both patella and lateral tibia. The reduction in PG-depletion for A03 in lateral tibia in experiment 2 is significant (p=0.02). The two FIGS. 9 and 10 should be compared to the image analysis of patella (corresponds to FIG. 9, patella analysed with arbitrary scale) where the result is shown in FIG. 8 (P=0.015).

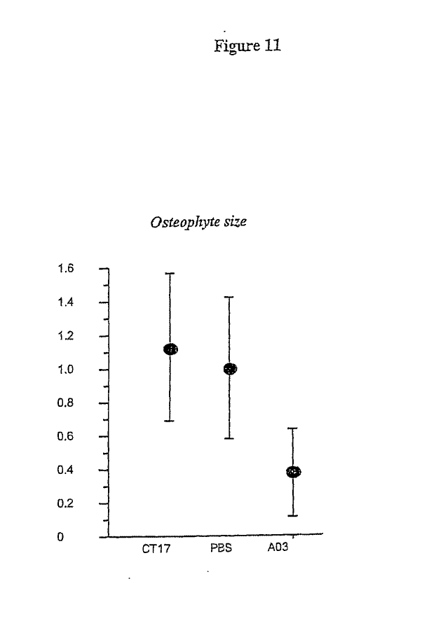

FIG. 11 demonstrates the effect of treatment with the anti-α11 integrin antibody on osteophyte size. As could be seen in the figure, a reduction of the osteocyte size is seen after treatment with the A03-antibody compared to treatment with control antibody (CT17).

### Conclusion

Treatment with the exemplary anti-α11 integrin antibody of the invention (A03) reduces inflammation of the joints of arthritic mice induced with Antigen-Induced Arthritis (AIA), measured as joint swelling and infiltration of inflammatory cells. In addition, the anti-α11 integrin treatment protects the cartilage from degradation, shown as reduction of proteoglycan degradation, in the AIA mice. Our results show a clear correlation between inflammation on day 3 (TC up take) and cartilage degradation (PG depletion) on day 15.

Current established treatments for RA reduces swelling (NSAIDS) or swelling and amount of infiltrating cells (anti-TNFα) in the same AIA model, but none have a protective effect on the cartilage (6-9). This suggests that anti-α11 integrin treatment has a great potential in RA therapy either as stand-alone therapy or in combination with other therapies.

### REFERENCES

1. van den Berg, W. B., M. W. Kruijsen, L. B. van de Putte, H. J. van Beusekom, M. van der Sluis-van der Pol, and W. A. Zwarts. 1981. Antigen-induced and zymosan-induced arthritis in mice: studies on in vivo cartilage proteoglycan synthesis and chondrocyte death. Br J Exp Pathol 62:308.
2. Lens, J. W., W. B. van den Berg, and L. B. van de Putte. 1984. Quantitation of arthritis by 99 mTc-uptake measurements in the mouse knee-joint: correlation with histological joint inflammation scores. Agents Actions 14:723.
3. Schubert, D., B. Maier, L. Morawietz, V. Krenn, and T. Kamradt. 2004. Immunization with glucose-6-phosphate isomerase induces T cell-dependent peripheral polyarthritis in genetically unaltered mice. J Immunol 172:4503.
4. Holmdahl, R., S. Carlsen, A. Mikulowska, M. Vestberg, U. Brunsberg, A.-S. Hansson, M. Sundvall, L. Jansson, and U. Pettersson. 1998. Genetic analysis of murine models for rheumatoid arthritis. In Human Genome Methods. K. W. Adolpho, ed. CRC press, New York: 215.
5. Mankin, H. J., H. Dorfman, L. Lippiello, and A. Zarin. 1971. Biochemical and metabolic abnormalities in articular cartilage from osteo-arthritic human hips. II. Correlation of morphology with biochemical and metabolic data. J Bone Joint Surg Am 53:523.
6. de Vries, B. J., W. B. van den Berg, E. Vitters, and L. B. van de Putte. 1988. Effect of nonsteroidal antiinflammatory drugs on cartilage destruction in antigen induced arthritis in mice. Scand J Rheumatol Suppl 77:23.
7. de Vries, B. J., and W. B. van den Berg. 1989. Impact of NSAIDS on murine antigen induced arthritis. I. Investigation of antiinflammatory and chondroprotective effects. J Rheumatol Suppl 18:10.
8. de Vries, B. J., and W. B. van den Berg. 1990. Impact of NSAIDS on murine antigen induced arthritis. II. A light microscopic investigation of antiinflammatory and bone protective effects. J Rheumatol 17:295.
9. van de Loo, A. A., O. J. Arntz, A. C. Bakker, P. L. van Lent, M. J. Jacobs, and W. B. van den Berg. 1995. Role of interleukin 1 in antigen-induced exacerbations of murine arthritis. Am J Pathol 146:239.

### Example III

### Effect of Exemplary Antibody of the Invention on Glucose-6-Phosphate-Induced Arthritis

The purpose of the study was to test the effect of an exemplary whole IgG antibody, designated ‘A03’, in DBA/1 mouse.

### Materials and Methods

### Testing Facility

Cartela AB, Scheelvagen 22, 223 63 Lund, Sweden.

### Experimental Design

The experimental protocol is shown in Tables 5 and 6 below.

TABLE 5

| Day | Procedure |
|---|---|
| 0 | Immunization of mice with hG6PI |
| 7, 10, 13, 16 | Administration of anti-integrin antibodies |
| 7 to 18 | Scoring of inflammation |
| 18 | Sacrifice of animals, bleeding, taking tissues for histology |

TABLE 6

| | | | Dose total |
|---|---|---|---|
| Group | Test drug | Batch | (μg/mouse) |
| 1 (control | IgG4 CT17 | CAR 141, Peak 2 run 4 + 5 | 880 |
| antibody) | | | |
| 2 | TNFR-p75- | Enbrel, Vnr 566661 | 880 |
| | IgFc | | |
| 3 | IgG4 A03 | PID 3640 | 880 |

### Animals

Female DBA1 mice were obtained from Taconic M&B, Denmark. The mice are fed with RMI Expanded (special Diet Services Ltd, UK) and water ad libitum. The mice are allowed an acclimatization period to he new environment for at least one week. Water and cages are changed 2 times per week. Food is completed when needed. The mice are stored 7 mice/Mac3 cage with enhanced lid.

### Antigen

Recombinant human glucose-6-phosphate isomerase (G6PI) was purchased from Kamradt (Deutches Rheumaforschungszentrum Berlin, Berlin, Germany), produced as previously described (3).

### Induction of Glucose-6-Phosphate Isomerase-Induced Arthritis

Mice were immunised with 200 μg recombinant human G6PI emulsified in a ration of 1:1 in CFA (Difco, Detroit, Mich., USA). Injection was given s.c. at the base of the tail with a volume of 100 μl (3).

### Administration of Test Drug

Antibodies were stored at 4° C. Antibodies were dissolved in sterile PBS and stored under sterile conditions at 4° C. Antibody administration was performed on day 7, 10, 13, 16 after injection of hG6PI. The antibodies were given i.p. 200 μl/mouse. Administration of the antibodies was always performed at the same time of the day −/+1 hr. A total of 880 μg antibody was given per mouse in the following scheme:

TABLE 7

| Day | microgram antibody per mouse |
|---|---|
| 0 | injection of hG6PI |
| 7 | 280 |
| 10 | 200 |
| 13 | 200 |
| 16 | 200 |

### Scoring Inflammation

Mice were visually scored for using an extended scoring protocol ranging from 1 to 15 for each paw, allowing a maximum score of 60 per mouse, arthritis as previously described (4). Each arthritic (red and swollen) toe and knuckle was scored as 1, whereas an affected ankle was scored as 5.

### Histology

Back paws and ankles were fixed in phosphate buffered formalin (pH 7.4) for 3 days, decalcified in EDTA and subsequently embedded in paraffin wax. Semi serial 5 μm sections were cut on a microtome. Front paws were decalcified in EDTA and then embedded in OCT and frozen. Semi serial 5 μm sections were cut on cryostat.

### Scoring of Cartilage Damage and Bone Destruction

Paraffin sections of back paws and ankles were stained with haematoxylin and eosin (H&E) or by Safranin-O, Sections were graded for proteoglycan depletion using the Manking grading system (5) and in addition also for osteophyte formation and bone destruction by light microscopy. Proteoglycan depletion was also scored as loss of red staining using digital image analysis.

### Immunostaining

Cryosections were allowed to warm up to room temperature then fixed in acetone for 10 minutes at −20° C. Slides were washed in PBS for 3×5 minutes, and were then incubated with hyaluronidase (2 mg/ml in PBS; Sigma), at 37° C. for 30 minutes. A second 3×5 minutes wash in PBS was performed before incubating sections with 4% goat serum (Sigma, G-9023) diluted in PBS for 30 minutes to block. Sections were incubated with biotinylated IgG4-anti-α11 integrin Ab diluted 1:100 (=20 ug/ml) in 4% goat serum in PBS, for 60 minutes at room temperature, and then washed in PBS for 3×5 minutes. Sections were then incubated for another 60 minutes with fluorescence (Alexa Fluor 448 or Cy3) conjugated Streptavidin. After additional wash in PBS for 3×5 minutes, slides were mounted with Vectashield, and observed under a fluorescence microscope.

### Results

Treatment with Antibody Against α11-Integrin Reduced Incidence and Severity of G6PI-Induced Arthritis in Mice

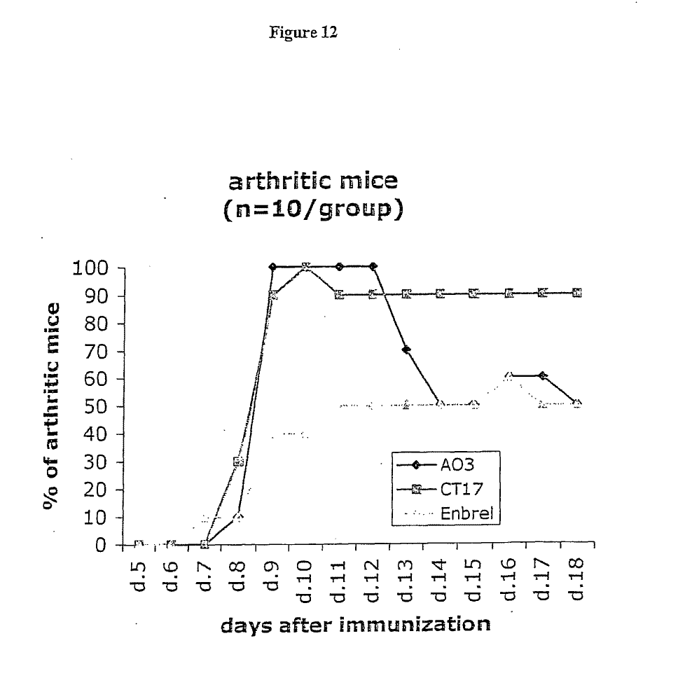

Immunization with recombinant human glucose-6-phosphate isomerase (rhG6PI) induces a poly-arthritis with severe swelling of both front and hind paws. Clinical signs of arthritis first appeared at day 7 after immunization, rapidly progressed and reached its maximum at day 9 (FIG. 12). The mice were sacrificed at day 18 after immunization.

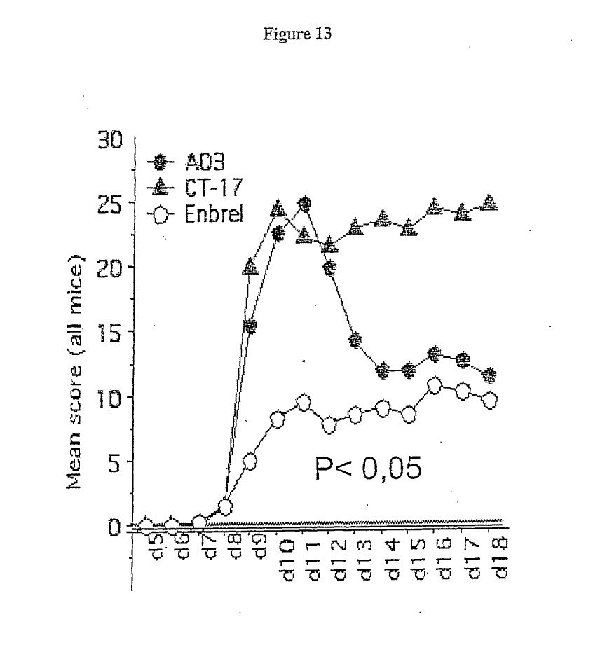

Treatment started at day 7 and was then given at day 10, 13, 16 after injection with a total of 880 μg of the test-antibody per mouse. The TNFR-p75-IgFc (Enbrel) was used as positive control. Treatment with Enbrel reduced the arthritis incidence to 50%, compared to 100% in the group treated with control antibody (CT17). The A03 treatment resulted in clear reduction of arthritis incidence and severity (FIGS. 12 and 13). The mice treated with A03 all developed arthritis (incidence 100% day 9) after which 50% of the mice recovered (p<0.05) (FIG. 13) and the incidence was reduced to the same level as that of the Enbrel treated group (p=0.051) (FIG. 12). FIG. 13 demonstrates the severity of arthritis measured as mean of arthritis scores of the individuals in each group.

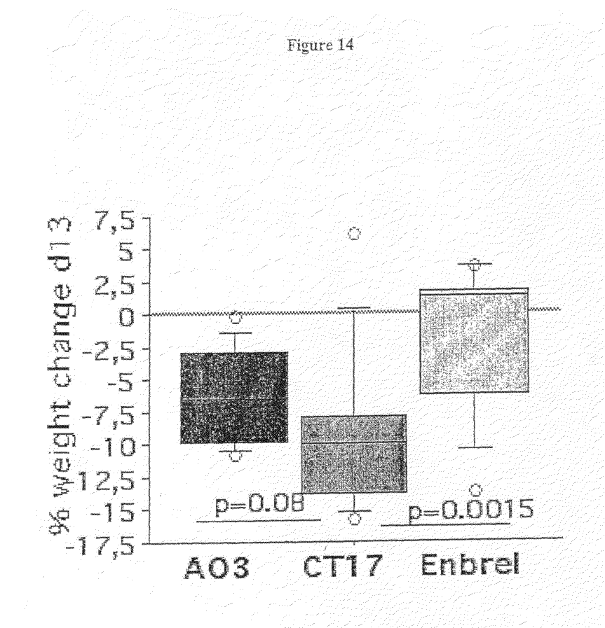

During the arthritis experiment the mice was weighted as a measurement of their general disease status. The A03 treated mice showed a clear, although not significant, less reduction of weight compared to the control treated mice (p=0.08) (FIG. 14).

Table 8 below demonstrates the incidence (frequency) of arthritis developed in each group tested in the experiments.

TABLE 8

| Treatment | Healthy | Sick | Totals |
|---|---|---|---|
| A03 | 0 | 10 | 10 |
| CT17 | 0 | 10 | 10 |
| Enbrel | 5 | 5 | 10 |
| Totals | 5 | 25 | 30 |

### Conclusion

Treatment with the anti-α11 integrin antibody (A03) reduces inflammation of the joints of arthritic mice in the poly-arthritic model glucose-6-phosphate isomerase (rhG6PI) induced arthritis. Using a treatment scheme where the test antibody was administrated at the day of onset and the subsequently every third day until termination of the experiment, one may demonstrate that the A03 treatment resulted in recovery of 50% of the arthritic mice. Incidence of arthritis was reduced from 100 to 50% in the test group, down to the level of incidence with Enbrel treatment.

### REFERENCES

1. van den Berg, W. B., M. W. Kruijsen, L. B. van de Putte, H. J. van Beusekom, M. van der Sluis-van der Pol, and W. A. Zwarts. 1981. Antigen-induced and zymosan-induced arthritis in mice: studies on in vivo cartilage proteoglycan synthesis and chondrocyte death. Br J Exp Pathol 62:308.
2. Lens, J. W., W. B. van den Berg, and L. B. van de Putte. 1984. Quantitation of arthritis by 99 mTc-uptake measurements in the mouse knee-joint: correlation with histological joint inflammation scores. Agents Actions 14:723.
3. Schubert, D., B. Maier, L. Morawietz, V. Krenn, and T. Kamradt. 2004. Immunization with glucose-6-phosphate isomerase induces T cell-dependent peripheral polyarthritis in genetically unaltered mice. J Immunol 172:4503.
4. Holmdahl, R., S. Carlsen, A. Mikulowska, M. Vestberg, U. Brunsberg, A.-S. Hansson, M. Sundvall, L. Janson, and U. Pettersson. 1998. Genetic analysis of murine models for rheumatoid arthritis. In Human Genome Methods. K. W. Adolpho, ed. CRC press, New York: 215.
5. Mankin, H J, H. Dorfman, L. Lippiello, and A. Zarins. 1971. Biochemical and metabolic abnormalities in articular cartilage from osteo-arthritic human hips. II. Correlation of morphology with biochemical and metabolic data. J Bone Joint Surg Am 53:523.

### Example IV

### Generation of Monoclonal Antibodies Competing to Exemplary “A03” Antibody

The purpose of this example is to generate mouse monoclonal antibodies competing to the exemplary “A03” antibody using hybridoma technology.

### Materials and Methods

### Immunization Protocol

HEK cell produced recombinant protein is used for immunization. The protein contains the upper-lower calf domain of human alpha11 (E-ULCD) to direct the antibody response against the stalk region of α11 integrin.

### Sequence of E-ULCD is

[SEQ ID NO: 19]
SDGSIECVNEERRLQKQVCNVSYPFFRAKAKVAFRLDFEFSKSIFLHHLE
IELAAGSDSNERDSTKEDNVAPLRFHLKYEADVLFTRSSSLSHYEVKLN
SSLERYDGIGPPFSCIFRIQNLGLFPIHGMMMKITIPIATRSGNRLLKLR
DFLTDEANTSCNIWGNSTEYRPTPVEEDLRRAPQLNHSNSDVVSINCNIR
LVPNQEINFHLLGNLWLRSLKALKYKSMKIMVNAALQRQFHSPFIFREED
PSRQIVFEISKQEDWQVP.

C57b1/6-alpha11 knock-out mice (as described in detail in WO 03/101497, incorporated herein by reference) were immunized.

Blood was drawn at indicated time points to be able to follow the immune response via FACS analysis. The following immunization scheme was applied:
Day 0 Day 0 blood for FACS analysis was drawn
   α11 Stalk protein (species of the alpha11 sequence is human, described in Veiling et al. J. Biol. Chem. 1999, 274:25735-25742.) (Cartela AB, batch#53; 070509; pooled peak 1+2, E-ULCD) (80 μg/mouse) was emulsified in CFA (Complete Freuds Adjuvant, Sigma) by sonication and injected at the base of the tail of four C57b1/6-α11ko mice.
Day 14 Blood was drawn for FACS analysis
   Booster immunization with alpha11 stalk (E-ULCD) (50 μg/mouse) in IFA (incomplete Freuds Adjuvant, Sigma).
Day 22 Blood was drawn for FACS analysis
Day 24 NSO fusion partners were expanded for fusion
Day 31 Pre-fusion i.p. boost with α11 stalk (batch #53 E-ULCD; 20 mM Tris, 1 mM Ca2+, Batch I) (50 μg/mouse) in PBS.
Day 34 B-cell fusion with the myeloma cell line NSO (ECACC)
   Fusion with NSO Cells Day 34 (070625)

Spleens of three C57b1/6-α11ko mice were used for fusions.

Single cell suspensions were made and erythrocytes were lysed using 9 ml sterile ddH2O (10 sec) before adding 1 ml 10×PBS to stop the lysis.

Cells were washed with serum-free D-MEM (2 times) and mixed with aliquots of washed NSO cells in a 50 ml Falcon tube (to remove free proteins which can be toxic during fusion with PEG). The pellets were kept at 37 C in a water bath. 1 ml 50% PEG (1500 MW) in D-MEM at 37 C was added drop by drop under stirring, using glass pipettes over a 1 min time period.

2 ml pre-warmed serum free D-MEM was added under a period of 2 min.

7 ml pre-warmed serum free D-MEM was added drop by drop over a period of 3 min.

Cells were centrifuged 5 min at 500×g.

Discharged supernatant and added pre-warmed complete D-MEM (Pyruvat, 10% FCS, PEST). Nr1 40 ml in 3 plates), Nr3 50 ml in 4 plates), Nr10 50 ml in 4 plates. Cells rested at RT for 10 min before adding approx. 100 μl of cells (1 drop per well) with 10 ml pipette to 96-well cell culture plates.

The fusion was performed according to the protocol in ‘Current Protocols in Immunology, Unit 2.5, “Production of monoclonal antibodies” (ISBN ISSBN: 1934-3671, Wiley, incorporated here in by reference.)
Day 35 Added 100 μl 2×HAT (hypoxantin-aminopterin-tymidin, Apoteket, Sweden) medium to wells for selection
Day 38 Remove toxic supernatant from dying cells, added fresh HT medium (hypoxantin-tymidin, Apoteket, Sweden)
Day 44 Remove 100 μl medium for FACS screening and added HT medium to the wells.

### Identification of Stalk Specific Hybridoma Through FACS Screens

Supernatants from wells containing the candidate hybridomas are tested for reactivity to the stalk region using flow cytometry (FACS). The clones are further characterized for their stalk reactivity and unwanted possible cross reactivity of the anti-alpha11 integrin stalk candidate hybridomas

### Characterization of Stalk Specific Hybridomas:

Unwanted cross reactivity of the anti α11-integrin stalk candidate hybridoma to the related α10-integrin is tested using α10-integrin transfected HEK and α10-integrin transfected C2C12 cells. Cross reactivity to the corresponding stalk from other species, i.e. mouse is tested using mouse α11-integrin transfected C2C12 cells.

### Subcloning of α11-Stalk Hybridomas

FACS-positive cells are subcloned using a limiting dilution method (approx. 0.3 cells/well). Rat thymocytes are used as feeder cells to ensure hybridoma survival. Hybridoma cells are frozen down during different subcloning stages.

### Generation of Antibodies to the Full Length Human Alpha11 Integrin.

Generation of antibodies to the full length human alpha11 integrin. Said full length alpha 11 integrin protein is affinity purified from human alpha11-transfected HEK cell lysates, using a polyclonal antiserum specific for the cytoplasmatic tail (as described in WO00/75187).

### Immunization Protocol: Full-Length α11-Integrin Project

Day 0 4 female C57b1/6-alpha11ko mice were used for immunization with 40 μg per mouse of full-length alpha11 protein (batch#070717) in CFA at the base of the tail.
   Blood was taken for FACS analysis.
Day 13 Day 13 blood was taken for FACS analysis
Day 16 Boost with full-length alpha11 protein (batch#070717) 10 μg/mouse in IFA
Day 26 Boost with a mix of 10 μg/mouse full-length alpha11 integrin and 40 μg stalk alpha11 protein (batch #53 E-ULCD)/mouse in PBS i.p.

Day 29 Fusion of spleen and lymph node cells with NSO cells Tail tips for DNA and serum was saved

Fusion with NSO Cells at Day 29

Spleens and cells from draining lymph nodes (LN) of three immunized alpha11ko mice were used for fusion.

Single cell suspension was made and erythrocytes of the spleen cells were lysed using 0.84% ammonium chloride.

Cells were washed with serum-free D-MEM (2 times) and mixed with aliquots of washed NSO cells in a 50 ml Falcon tube. The pellet was kept at 37 C in a water bath.

1 ml 50% PEG 1500 MW in DMEM at 37 C was added under stirring drop by drop over a 1 min time period. 2 ml pre-warmed serum free DMEM was added dropwise under a period of 2 min. 7 ml pre-warmed serum free DMEM was added drop by drop over a period of 3 min.

Cells were centrifuged 5 min at 500×g. Supernatants were discharged before adding pre-warmed complete DMEM (Pyruvat, 10% FCS, PS) to LN and spleen preparations; for Nr.1 in 50 ml, for Nr.3 in 50 ml, for Nr.10 in 50 ml. One drop (approx. 100 μl) per well were added to 4.5 plates per fusion. 100 μl 2×HAT medium was added 4 hours later.

The fusion was performed according to the protocol in ‘Current Protocols in Immunology, Unit 2.5, “Production of monoclonal antibodies” (ISBN ISSBN: 1934-3671, Wiley, incorporated here in by reference.)
Day 100 μl medium was removed and HT medium added to the wells.

Supernatants are taken at day 11 after fusion for FACS screening of positive well as described above. After identifying positive wells, supernatants were tested for cross reactivity between human and mouse alpha11 integrins (hybridoma screen using C2C12 wt cells and cells transfected with human alpha11, and alpha10-integin.

Hybridoma cells were subcloned (0.3 cells/well) in BM-condimed H1 supplemented medium (Roche, Del.). Wells with one colony only growing were selected for specificity screening.

Screening for Competition with Exemplary Antibody “A03”

Further, newly generated clones are tested for its ability to block the binding of the exemplary antibody “A03”. A FACS based method is used. Human alpha11-integrin transfected C2C12 cells are incubated with purified newly generated antibodies in a dilution series of about 1.6 up to 1000 ng/ml to occupy their epitope on the alpha11-integrin expressed on the cell surface. In a second step biotinylated exemplary antibody A03 is added. If these antibodies share an epitope region, the binding of the human IgGs will be reduced.

1.-109. (canceled)

110. An antibody or fragment thereof capable of competing for binding to an integrin α11 subunit, or a heterodimer thereof, with an scFv molecule having the amino acid sequence of SEQ ID NO: 13, and wherein the antibody or fragment does not comprise all of the amino acid sequences of SEQ ID NOs: 1-6.

111. The antibody or fragment of claim 110, wherein the antibody or fragment is capable of binding to the same epitope as an scFv molecule having the amino acid sequence of SEQ ID NO: 13.

112. The antibody or fragment of claim 110, wherein the antibody or fragment is capable of binding to an epitope distinct from that to which an scFv molecule having the amino acid sequence of SEQ ID NO: 13 binds.

113. The antibody or fragment of claim 110, wherein the antibody or fragment is capable of modulating the degradation of collagen.

114. The antibody or fragment of claim 113, wherein the antibody or fragment is capable of inhibiting the degradation of collagen.

115. The antibody or fragment of claim 113, wherein the antibody or fragment is capable of modulating the degradation of collagen in vivo.

116. The antibody or fragment of claim 110, wherein the antibody or fragment has efficacy in the treatment of an inflammatory condition.

117. The antibody or fragment of claim 116, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis and/or an autoimmune chronic inflammatory disease.

118. The antibody or fragment of claim 117, wherein the inflammatory condition is rheumatoid arthritis.

119. The antibody or fragment of claim 117, wherein the inflammatory condition is osteoarthritis.

120. A pharmaceutical composition comprising the antibody or fragment of claim 110 and a pharmaceutically acceptable excipient, diluent or carrier.

121. A method for treating an inflammatory condition, comprising administering to an individual in need thereof an effective amount of the antibody or fragment of claim 110.

122. The method of claim 121, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

123. The method of claim 122, wherein the inflammatory condition is an arthritic disease.

124. The method of claim 123, wherein the arthritic disease is rheumatoid arthritis or osteoarthritis.

125. A method for diagnosing or prognosing an inflammatory condition, comprising administering to an individual in need thereof an effective amount of the antibody or fragment of claim 110.

126. The method of claim 125, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

127. The method of claim 126, wherein the inflammatory condition is an arthritic disease.

128. The method of claim 127, wherein the arthritic disease is rheumatoid arthritis or osteoarthritis.

129. A method for detecting or imaging cells expressing an integrin α11 subunit, or a heterodimer thereof, associated with an inflammatory condition, in the body of an individual, the method comprising administering to the individual an effective amount of the antibody or fragment thereof of claim 110.

130. The method of claim 129, further comprising the step of detecting the location of the antibody or fragment thereof in the individual.

131. The method of claim 129, wherein the antibody or fragment binds to an integrin α11 subunit, or a heterodimer thereof, which is localised on the surface of a cell.

132. The method of claim 131, wherein the cell is a chondrocyte or a fibroblast.

133. The method of claim 129, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

134. The method of claim 133, wherein the inflammatory condition is an arthritic disease.

135. The method of claim 134, wherein the arthritic disease is rheumatoid arthritis or osteoarthritis.

136. A method for monitoring the progression of an inflammatory condition in an individual, the method comprising:
(a) providing a sample of cells collected from the individual at a first time point and measuring the amount of integrin α11 subunit protein therein using the antibody or fragment of claim 110,
(b) providing a sample of cells collected from the individual at a second time point and measuring the amount of integrin α11 subunit protein therein using the antibody or fragment of claim 110, and
(c) comparing the amount of integrin α11 subunit protein measured in steps (a) and (b),
wherein an increased amount of integrin α11 subunit protein measured in step (b) as compared to step (a) is indicative of a progression in the inflammatory condition.

137. The method of claim 136, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

138. The method of claim 137, wherein the inflammatory condition is an arthritic disease.

139. The method of claim 138, wherein the arthritic disease is rheumatoid arthritis or osteoarthritis.

140. A method for identifying cells associated with an inflammatory condition, the method comprising measuring the amount of integrin α11 subunit protein in a test sample of cells using the antibody or fragment of claim 110 and comparing the amount of integrin α11 subunit protein in the test sample with the amount of integrin α11 subunit protein in a positive and/or negative control.

141. The method of claim 140, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

142. The method of claim 141, wherein the inflammatory condition is an arthritic disease.

143. The method of claim 142, wherein the arthritic disease is rheumatoid arthritis or osteoarthritis.

144. A method for identifying a candidate compound for treating an inflammatory condition, the method comprising:
(a) providing a test compound;
(b) testing the ability of the compound to compete for binding to an integrin all subunit, or a heterodimer thereof, with the antibody or fragment of claim 110,
wherein the test compound is identified as a candidate compound for treating an inflammatory condition if it is able to compete for binding to the integrin α11 subunit, or heterodimer thereof.

145. The method of claim 144, comprising the step of testing the ability of the compound to modulate the degradation of collagen.

146. The method of claim 144, comprising the step of testing the ability of the compound to inhibit the degradation of collagen.

147. The method of claim 144, wherein the testing step(s) are performed in vivo.

148. The method of claim 144, wherein the test compound comprises a polypeptide.

149. The method of claim 144, wherein the test compound comprises an antibody or fragment thereof.

150. The method of claim 149, wherein the test compound is an intact antibody.

151. The method of claim 149, wherein the test compound is a Fv fragment, a Fab-like fragment, a single antibody chain, a single variable domain or a domain antibody.

152. A nucleic acid molecule encoding the antibody or fragment of claim 110.

153. A vector comprising the nucleic acid molecule of claim 152.

154. A recombinant host cell comprising the nucleic acid molecule of claim 152.

155. A method for producing the antibody or fragment of claim 110, comprising culturing a host cell comprising a nucleic acid molecule encoding the antibody or fragment under conditions which permit expression of the encoded antibody or fragment.

156. A kit comprising the pharmaceutical composition of claim 120.

157. The antibody or fragment of claim 110, wherein the antibody comprises an intact antibody.

158. The antibody or fragment of claim 110, wherein the antibody or fragment is a Fv fragment, a Fab-like fragment, a single antibody chain, a single variable domain or a domain antibody.

159. The antibody or fragment of claim 110, wherein the antibody or fragment is a scFv.

160. The antibody or fragment of claim 110, wherein the antibody is a recombinant antibody.

161. The antibody or fragment of claim 110, wherein the antibody is a monoclonal antibody.

162. The antibody or fragment of claim 110, wherein the antibody or fragment is human or humanised.

163. The antibody or fragment of claim 110, wherein the antibody or fragment comprises a therapeutic and/or detectable moiety.

164. An epitope defined by an antibody or fragment thereof capable of binding to an integrin α11 subunit, or a heterodimer thereof, wherein the antibody or fragment comprises the amino acid sequences of SEQ ID NOs: 1-6.

165. The epitope of claim 164 comprising the amino acid sequence of SEQ ID NO: 15, or a fragment or variant thereof.

166. An isolated polypeptide comprising the amino acid sequence of SEQ ID NO: 15, or a fragment, fusion or variant thereof.

167. The isolated polypeptide of claim 172, wherein the polypeptide is capable of stimulating an immune response.

168. The isolated polypeptide of claim 172, wherein the polypeptide is capable of binding to an antibody or a fragment thereof with binding specificity for an integrin α11 subunit, or a heterodimer thereof.

169. A method for treating an inflammatory condition, comprising administering to an individual in need thereof an effective amount of an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13.

170. The method of claim 169, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

171. The method of claim 170, wherein the inflammatory condition is an arthritic disease.

172. The method of claim 171, wherein the arthritic disease is rheumatoid arthritis or osteoarthritis.

173. A method for diagnosing or prognosing an inflammatory condition, comprising administering to an individual in need thereof an effective amount of an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13.

174. The method of claim 173, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

175. The method of claim 174, wherein the inflammatory condition is an arthritic disease.

176. The method of claim 175, wherein the arthritic disease is rheumatoid arthritis or osteoarthritis.

177. A method for detecting or imaging cells expressing an integrin α11 subunit, or a heterodimer thereof, associated with an inflammatory condition, in the body of an individual, the method comprising administering to the individual an effective amount of an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13.

178. The method of claim 177, further comprising the step of detecting the location of the antibody or fragment thereof in the individual.

179. The method of claim 177, wherein the antibody or fragment binds to an integrin α11 subunit, or a heterodimer thereof, which is localised on the surface of a cell.

180. The method of claim 179, wherein the cell is a chondrocyte or a fibroblast.

181. The method of claim 177, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

182. The method of claim 181, wherein the inflammatory condition is an arthritic disease.

183. The method of claim 182, wherein the arthritic disease is rheumatoid arthritis or osteoarthritis.

184. A method for monitoring the progression of an inflammatory condition in an individual, the method comprising:
(a) providing a sample of cells collected from the individual at a first time point and measuring the amount of integrin α11 subunit protein therein using an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13,
(b) providing a sample of cells collected from the individual at a second time point and measuring the amount of integrin α11 subunit protein therein using an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13, and
(c) comparing the amount of integrin α11 subunit protein measured in steps (a) and (b),
wherein an increased amount of integrin α11 subunit protein measured in step (b) as compared to step (a) is indicative of a progression in the inflammatory condition.

185. The method of claim 184, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

186. The method of claim 185, wherein the inflammatory condition is an arthritic disease.

187. The method of claim 186, wherein the arthritic disease is rheumatoid arthritis or osteoarthritis.

188. A method for identifying cells associated with an inflammatory condition, the method comprising
(a) measuring the amount of integrin α11 subunit protein in a test sample of cells using an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13, and
(b) comparing the amount of integrin α11 subunit protein in the test sample with the amount of integrin α11 subunit protein in a positive and/or negative control.

189. The method of claim 188, wherein the inflammatory condition is an arthritic disease, joint inflammation, inflammation-induced cartilage destruction, chronic inflammatory bowel disease (IBD), Crohn's disease, ulcerative colitis, peridontitis, psoriasis, asthma, systemic lupus erythematosus, multiple sclerosis, ankylosing spondylitis, psoriatic arthritis and/or an autoimmune chronic inflammatory disease.

190. The method of claim 189, wherein the inflammatory condition is an arthritic disease.

191. The method of claim 190, wherein the arthritic disease is rheumatoid arthritis or osteoarthritis.

192. A method for identifying a candidate compound for treating an inflammatory condition, the method comprising:
(a) providing a test compound;
(b) testing the ability of the compound to compete for binding to an integrin α11 subunit, or a heterodimer thereof, with an antibody or a fragment thereof capable of binding to an integrin α11 subunit, or a heterodimer thereof, wherein the antibody or fragment comprises the amino acid sequences of SEQ ID NOs: 1-6; a heavy chain variable region comprising CDRs with the amino sequences of SEQ ID NOs: 1-3; a heavy chain variable region with the amino acid sequence of SEQ ID NO: 9; a light chain variable region comprising CDRs with the amino sequences of SEQ ID NOs: 4-6; a light chain variable region with the amino acid sequence of SEQ ID NO: 10; a heavy chain comprising the amino acid sequence of SEQ ID NO: 11; a light chain comprising the amino acid sequence of SEQ ID NO: 12; or the amino acid sequence of SEQ ID NO: 13,
wherein the test compound is identified as a candidate compound for treating an inflammatory condition if it is able to compete for binding to the integrin α11 subunit, or heterodimer thereof.

193. The method of claim 192, comprising the step of testing the ability of the compound to modulate the degradation of collagen.

194. The method of claim 192, comprising the step of testing the ability of the compound to inhibit the degradation of collagen.

195. The method of claim 192, wherein the testing step(s) are performed in vivo.

196. The method of claim 192, wherein the test compound comprises a polypeptide.

197. The method of claim 192, wherein the test compound comprises an antibody or fragment thereof.

198. The method of claim 197, wherein the test compound is an intact antibody.

199. The method of claim 197, wherein the test compound is a Fv fragment, a Fab-like fragment, a single antibody chain, a single variable domain or a domain antibody.


SEQUENCE LISTING (19 sequences)

SEQ ID NO: 1 - protein, 14 aa, Artificial Sequence
  anti-integrin alpha-11 antibody or antigen binding fragment
FSRYYMHWVRQVPG

SEQ ID NO: 2 - protein, 19 aa, Artificial Sequence
  anti-integrin alpha-11 antibody or antigen binding fragment
SGVSWNGSRTHYADSVKGR

SEQ ID NO: 3 - protein, 12 aa, Artificial Sequence
  anti-integrin alpha-11 antibody or antigen binding fragment
ARVSGDGYNFGA

SEQ ID NO: 4 - protein, 15 aa, Artificial Sequence
  anti-integrin alpha-11 antibody or antigen binding fragment
CTGSSSNIGAGYDVH

SEQ ID NO: 5 - protein, 7 aa, Artificial Sequence
  anti-integrin alpha-11 antibody or antigen binding fragment
GYNERPS

SEQ ID NO: 6 - protein, 13 aa, Artificial Sequence
  anti-integrin alpha-11 antibody or antigen binding fragment
CAAWDDSLSGHVV

SEQ ID NO: 7 - protein, 364 aa, Artificial Sequence
  epitope fragment of integrin alpha-11 subunit
WNGCNEDEHCVPDLVLDARSDLPTAMEYCQRVLRKPAQDCSAYTLSFDTTVFIIESTRQRVAVEATLENRGENAYSTVLNISQSANLQFASLIQKEDSDGSIECVNEERRLQKQVCNVSYPFFRAKAKVAFRLDFEFSKSIFLHHLEIELAAGSDSNERDSTKEDNVAPLRFHLKYEADVLFTRSSSLSHYEVKLNSSLERYDGIGPPFSCIFRIQNLGLFPIHGMMMKITIPIATRSGNRLLKLRDFLTDEANTSCNIWGNSTEYRPTPVEEDLRRAPQLNHSNSDVVSINCNIRLVPNQEINFHLLGNLWLRSLKALKYKSMKIMVNAALQRQFHSPFIFREEDPSRQIVFEISKQEDWQVP

SEQ ID NO: 8 - protein, 23 aa, Artificial Sequence
  epitope fragment of integrin alpha-11 subunit
EYCQRVLRKPAQDCSAYTLSFDT

SEQ ID NO: 9 - protein, 119 aa, Artificial Sequence
  anti-integrin alpha-11 antibody or antigen binding fragment
EVQLLESGGGLVQPGGSLRLSCAASGFTFSRYYMHWVRQVPGKGLEWVSGVSWNGSRTHYADSVKGRFTISRDNSKNTLYLQMNSLRAEDTAVYYCARVSGDGYNFGAWGQGTLVTVSS

SEQ ID NO: 10 - protein, 113 aa, Artificial Sequence
  anti-integrin alpha-11 antibody or antigen binding fragment
QSVLTQPPSASGTPGQRVTISCTGSSSNIGAGYDVHWYQQLPGTAPKLLIYGYNERPSGVPDRFSGSKSGTSASLAISGLRSEDEADYYCAAWDDSLSGHVVFGGGTKLTVLG

SEQ ID NO: 11 - protein, 446 aa, Artificial Sequence
  anti-integrin alpha-11 antibody or antigen binding fragment
EVQLLESGGGLVQPGGSLRLSCAASGFTFSRYYMHWVRQVPGKGLEWVSGVSWNGSRTHYADSVKGRFTISRDNSKNTLYLQMNSLRAEDTAVYYCARVSGDGYNFGAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 218 aa, Artificial Sequence
  anti-integrin alpha-11 antibody or antigen binding fragment
QSVLTQPPSASGTPGQRVTISCTGSSSNIGAGYDVHWYQQLPGTAPKLLIYGYNERPSGVPDRFSGSKSGTSASLAISGLRSEDEADYYCAAWDDSLSGHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 247 aa, Artificial Sequence
  anti-integrin alpha-11 antibody or antigen binding fragment
EVQLLESGGGLVQPGGSLRLSCAASGFTFSRYYMHWVRQVPGKGLEWVSGVSWNGSRTHYADSVKGRFTISRDNSKNTLYLQMNSLRAEDTAVYYCARVSGDGYNFGAWGQGTLVTVSSGGGGSGGGGSGGGGSQSVLTQPPSASGTPGQRVTISCTGSSSNIGAGYDVHWYQQLPGTAPKLLIYGYNERPSGVPDRFSGSKSGTSASLAISGLRSEDEADYYCAAWDDSLSGHVVFGGGTKLTVLG

SEQ ID NO: 14 - protein, 22 aa, Artificial Sequence
  anti-integrin alpha-11 antibody or antigen binding fragment
EQKLISEEDLSGSAAAHHHHHH

SEQ ID NO: 15 - protein, 267 aa, Artificial Sequence
  epitope fragment of integrin alpha-11 subunit
SDGSIECVNEERRLQKQVCNVSYPFFRAKAKVAFRLDFEFSKSIFLHHLEIELAAGSDSNERDSTKEDNVAPLRFHLKYEADVLFTRSSSLSHYEVKLNSSLERYDGIGPPFSCIFRIQNLGLFPIHGMMMKITIPIATRSGNRLLKLRDFLTDEANTSCNIWGNSTEYRPTPVEEDLRRAPQLNHSNSDVVSINCNIRLVPNQEINFHLLGNLWLRSLKALKYKSMKIMVNAALQRQFHSPFIFREEDPSRQIVFEISKQEDWQVP

SEQ ID NO: 16 - DNA, 1338 bp, Artificial Sequence
  Heavy chain of anti-alpha11 whole IgG antibody (AO3)
gaggtgcagctgttggagtctgggggaggcttggtacagcctggggggtccctgagactctcctgtgcagcctctggattcaccttcagtcgttactacatgcactgggtccgccaagttccagggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgcgagagtaagtggagatggctacaattttggcgcctggggccagggtacactggtcaccgtgagctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 17 - DNA, 654 bp, Artificial Sequence
  Light chain of anti-alpha11 whole IgG antibody (A03)
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggttataatgagcggccctcaggggtccctgaccgattctctggctccaagtctggcacctcagcctccctggccatcagtgggctccggtccgaggatgaggctgattattactgtgcagcatgggatgacagcctgagtggtcatgtggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 18 - DNA, 741 bp, Artificial Sequence
  ScFv format of anti-alpha11 antibody (A03)
gaggtgcagctgttggagtctgggggaggcttggtacagcctggggggtccctgagactctcctgtgcagcctctggattcaccttcagtcgttactacatgcactgggtccgccaagttccagggaaggggctggagtgggtatcgggtgttagttggaatggcagtaggacgcactatgcagactctgtgaagggccgattcaccatctccagagacaattccaagaacacgctgtatctgcaaatgaacagcctgagagccgaggacactgccgtgtattactgtgcaagagtaagtggagatggctacaattttggcgcctggggccagggtacactggtcaccgtgagcagcggtggaggcggttcaggcggaggtggatccggcggtggcggatcgcagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatggttataatgagcggccctcaggggtccctgaccgattctctggctccaagtctggcacctcagcctccctggccatcagtgggctccggtccgaggatgaggctgattattactgtgcagcatgggatgacagcctgagtggtcatgtggtattcggcggaggaaccaagctgacggtcctaggt

SEQ ID NO: 19 - protein, 267 aa, Artificial Sequence; identical to SEQ ID NO: 15
  E-ULCD recombinant protein